(19) United States
(12) Patent Application Publication
(10) Pub. No.: US 2006/0036138 A1
(43) Pub. Date: Feb. 16, 2006

(54) Devices and methods of screening for neoplastic and inflammatory disease

(76) Inventors: Adam Heller, Austin, TX (US); Thomas Liebermann, Austin, TX (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/197,872

(22) Filed: Aug. 5, 2005

Related U.S. Application Data
(60) Provisional application No. 60/683,518, filed on May 20, 2005.
(60) Provisional application No. 60/599,752, filed on Aug. 6, 2004.

(51) Int. Cl. A61B 5/00

(52) U.S. Cl. 600/309

(57) ABSTRACT

Methods and devices are provided for evaluating the presence of disease in a patient. In particular, methods and devices are provided for screening patients for neoplastic and/or inflammatory disease. Such diseases are often indicated by the elevated level of a chemical compound associated with disease, such as nitric oxide (NO) and/or nitrogen dioxide (NO₂). Through measuring and/or estimating the chemical compound-concentration, such as by change in fluorescence, absorbance or reflectance, the methods and tools provided distinguish between patients who require further testing and/or treatment and those who do not. The methods and tools also provide information about the effectiveness of treatment, such as treatment to reduce inflammation or control of the growth of malignant tumors. These methods and devices are relatively inexpensive, easy to use, and provide other advantages.

### CROSS-REFERENCES TO RELATED APPLICATIONS

This application claims the benefit and priority of U.S. Provisional Patent Application No. 60/599,752 (Attorney Docket 021821-000500US), filed Aug. 6, 2004, and U.S. Provisional Patent Application No. 60/683,518 (Attorney Docket 021821-000520US), filed May 20, 2005, the full disclosures of which are hereby incorporated by reference for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

There is a need for clinically useful methods and tools for diagnosing pathological elevation in the concentration of nitric oxide (NO) in parts of the human body. NO is a recognized endothelium derived relaxing factor, a cardiovascular signaling molecule and a neurotransmitter. It is found in most, possibly in all, parts of the normal body but only at relatively low concentrations. For example, in the healthy epithelium of blood vessels in the skin and in the bowel, the typical NO concentration is between about 0.1 nM and about 10 nM. In healthy neuron-rich tissues typical NO concentration is about 10-200 nM.

NO concentration is, however, much higher in diseased tissues and organs, and is particularly high in inflamed and/or cancerous tissues and organs. In diseased tissues and organs, NO is a precursor of cytotoxic radicals like the carbonate radical anion (.CO₃⁻), nitrogen dioxide (.NO₂) and the hydroxyl radical (.OH). Pathogen and foreign body fighting macrophages and neutrophils, as well as cells of diseased, but usually not of healthy, tissues, express and, when stimulated by cytokines or chemokines, over-express the enzyme inducible nitric oxide synthase (iNOS). The enzyme (iNOS) catalyzes the reaction of arginine with oxygen, whereby NO is produced at a high rate. In diseased tissues and organs, particularly in inflamed and/or cancerous tissues and/or organs, the NO concentration is higher than that in the healthy tissue and/or organ, often by more than an order of magnitude. The increase is documented in more than one thousand publications, the titles and/or abstracts of which are accessible through searching MEDLINE using the combination of a term of the group (inflammatory or inflammation or neoplasia or tumor or carcinoma or sarcoma) with a term of the group (inducible nitric oxide synthase, or iNOS, or nitric oxide concentration). It is also documented in numerous patents.

NO Concentration in Diagnosis of Inflammatory Bowel Disease

J. Lundberg et al., Nature Clinical Practice (Gastroenterology and Hepatology), 2005, 2(2), 96-102 reported an increase by about an order of magnitude, in some cases even by two orders of magnitude, in the concentration of NO in the luminal gas of the bowel in inflammatory bowel disease. They determined the NO concentration using air filled balloons that were rectally inserted to about 10-15 cm depth. After equilibration with the luminal gas for about 20 min, the balloons were retrieved and their gas was analyzed for NO by chemiluminescence, the NO reacting with ozone (03) to produce excited, mostly infrared light emitting, NO₂. In healthy controls, the luminal NO concentration was 50-250 ppb. In people with active inflammatory bowel disease, the luminal NO concentration was 1000-50000 ppb. In people with non-inflammatory irritable bowel syndrome, the luminal NO concentration was 50-200 ppb. And in people with inactive inflammatory bowel disease, the luminal NO concentration was 50-500 ppb.

T. Ljung et al. Journal of pediatric gastroenterology and nutrition 2002, 34(3), 302-6 reported in children with active inflammatory bowel disease an increase in the rectal gas phase NO concentration by as much as two orders of magnitude. The NO concentration was 77±17 ppb in healthy children, 8,840±120 ppb in children with ulcerative colitis and 15,170±4,757 ppb in children with Crohn's disease. Children with non-active ulcerative colitis had rectal NO concentrations of 356±110 ppb and children with inactive Crohn's disease of 188±55 ppb. Their results showed that not only is the measurement of NO important in diagnosing disease, but that it is also an effective tool in determining the effectiveness of treatment of disease.

In related patents U.S. Pat. No. 6,063,027 and U.S. Pat. No. 6,183,416, K. Alving et al. describe diagnosis of inflammatory conditions in the intestinal canal by measuring the NO-concentration in the luminal gas of the bowel, preferably by the chemiluminescence of the excited NO₂ generated in the reaction of NO with ozone (O₃).

NO Concentration in Diagnosis of Colorectal Adenomas and Cancer

Inducible nitric oxide synthase (iNOS) is over-expressed in neoplasms, implying elevated NO concentrations. Importantly, it is particularly over-expressed in pre-cancerous adenomas (polyps) and in the early, easily operable, stages of colorectal cancer. Y. Kojima et al. J. Surg. Oncol. 1999, 70(4):222-9 reported high nitric oxide synthase expression and nitric oxide production in human colon carcinoma tissue; N. Yagihashi, Virchows Arch. 2000, 436(2):109-14 reported increased in situ expression of iNOS in human colorectal cancer. R. J. Bing et al., Clin Cancer Res. 2001, 7(11):3385-92 found increased expression of iNOS in human colon cancer tissue obtained during surgery. In a study of 25 cases, they observed an increase in immunoreactive iNOS in the tumor cells in 22 cases. M. H. Xu et al. World J Gastroenterol. 2003, 9(6): 1246-50 studied the role of iNOS expression in the aberrant crypt foci (ACF)-adenoma-carcinoma sequence. The immunoreactivity of iNOS significantly increased in the transition from hyperplastic ACF to dysplastic ACF. The expression of iNOS was high after transition from hyperplastic ACF to dysplastic ACF, adenoma and carcinoma. H. Cen et al., World J Gastroenterol. 2004, 10(21):3122-6 found increased expression of the iNOS gene in colon cancer tissues compared to normal colon tissue. The expression of iNOS was increased in 63% ( 22/35) of the patients studied.

Nitric oxide is formed by the iNOS catalyzed oxidation of arginine. N. Gupta et al. Biochim Biophys Acta 2005, 1741(1-2):215-2 report that the mRNA of the arginine transporter ATB(0,+), which is expressed at low levels in normal colon, increased 22.9+/−3.0-fold in colorectal cancer compared to normal tissue. The increase was evident in each of the 10 cases examined. iNOS mRNA increased 5.2+/−1.1-fold in cancer specimens. The changes in mRNA levels were associated with an increase in ATB(0,+), in iNOS, and in nitrotyrosylated proteins.

K. Nosho et al., Br. J. Cancer 2005, 92(7):1193-200 found that iNOS is one of the upregulated genes at the early stage of colorectal carcinogenesis. In tumor tissues it was over five times higher than those in matched normal tissues. K. M. Ropponen et al., Scand J. Gastroenterol. 2000, 35(11):1204-11 found in a study of 157 colorectal carcinoma patients that iNOS intensity and percentage of iNOS positive cells was moderate or intense in 37% of the tumors, but were higher in the still operable Dukes A and B adenocarcinomas of the colon or rectum than in the advanced-stage, often inoperable, Dukes C and D adenocarcinomas. Similarly, J. A. Lagares-Garcia et al., American surgeon, 2001, 67(7), 709-13 found elevated inducible nitric oxide synthase (iNOS) activity in 60% of the colon adenomas and in 20-50% of the adenocarcinomas.

NO Elevation in Gastric Cancers.

Significant elevation in iNOS and/or NO in gastric cancers was reported by L. Wang et al., Gastric Cancer 2005, 8(1):18-28; L. G. Li and H. M. Xu World J. Gastroenterol. 2005, 11(17):2539-44; B. Hazar et al., Hepatogastro-enterology 2005, 52(61):119-22; Y. Z. Wang et al. World J. Gastroenterol. 2005, 11 (1):46-50; M. Ichinoe et al., Histopathology 2004, 45(6):612-8; H. L. Li et al., World J. Gastroenterol. 2004, 10(13):1862-6; Z. Y. Song et al., World J Gastroenterol. 2004, 10(9):1250-5 and 2002, 8(4):591-5; N. Ilhan et al., World J. Gastroenterol. 2004, 10(8):1115-20; C. J. van der Woude et al., J. Clin. Pathol. 2003, 56(9):699-702; P. D. Khare et al., Anticancer Res. 2002, 22(4):2443-6; E. Bakan et al., Japan. J. Clin Oncol. 2002, 32(5):162-6; C. W. Feng et al., BMC Cancer, 2002, 2(1):8; C. Oldreive and C. Rice-Evans, Free Radical Res. 2001, 35(3):215-31; H. J. Son et al., J. Clin. Gastroenterol. 2001, 33(5):383-8; A. Rajnakova et al., Cancer Lett. 2001, 172(2):177-85; E. Koh et al., Cancer Lett. 1999, 146(2):173-80. A. Eroglu et al., British J. Cancer. 1999, 80(10):1630-4; and M. C. Symons et al., Free Radical Res. 1994, 21(4):197-202. iNOS elevation was reported in the pre-cancerous Helicobacter pylori infected stomach (G. Rieder et al. “Up-regulation of inducible nitric oxide synthase in Helicobacter pylori-associated gastritis may represent an increased risk factor to develop gastric carcinoma of the intestinal type” Int. J. Med. Microbiol. 2003, 293(6):403-12).

Screening for Need of Colonoscopy and/or Fecal DNA Assay

The annual number of colonoscopies in the US exceeds 14 million and their cost to society exceeds $ 20 billion. In people younger than about 50, without a family history of colorectal cancer, colonoscopy is often performed to differentiate between inflammatory bowel disease and irritable bowel syndrome. Only about one in ten patients undergoing colonoscopy has inflammatory bowel disease. Billions of dollars would be saved by screening the candidates for colonoscopy, differentiating between those who are more likely to have inflammatory bowel disease, and for whom colonoscopy is necessary, from those with irritable bowel syndrome, who do not require colonoscopy. Therefore, a simple an inexpensive test to provide such screening is desired and is an objective of which the present invention is directed.

In people older than about 50, and in younger people with a family history of colorectal cancer, colonoscopy costing more than $1,500, or fecal DNA assay, costing about $800, is performed additionally to screen for adenomas, also known as polyps, and/or for colorectal cancer, the second ranking cause of cancer-caused death in the US. The prevalence of colorectal cancer increases steeply with age, but even in 80+people, of an average age of 85, cancer is found only in about 1/16^{th} of the colonoscopies. Only about 1/10^{th} of the colonoscopies reveal adenomas in the 50+ population. Nearly ⅔^{rd} of the 50+ people, who should be screened for adenomas and for colorectal cancer, are not screened because of the cost and/or discomfort involved. Broader screening of the 50+ candidates for adenomas that could develop into colorectal cancer and for early, still curable/operable stage of colorectal cancer, and referral for colonoscopy of only those people with elevated colorectal NO concentration, measured with little discomfort and at low cost, would reduce the mortality of colorectal cancer.

iNOS Elevation in Other Cancers in or Near Other Gas Containing Spaces

iNOS elevation was reported also in cancer of the prostate (J. Wang et al. “Expression of inducible nitric oxide synthase in paired neoplastic and non-neoplastic primary prostate cell cultures and prostatectomy specimen” Urol. Oncol. 2003, 21(2): 117-22); and in tumors of the head and neck, (T. Umar et al. “Expression of inducible nitric oxide synthase in cutaneous adnexal tumors of the head and neck”, Int. J. Oral Maxillofacial. Surg. 2003, 32(5):534-8).

Some Relevant Properties of NO

NO is a gas at ambient temperature. Its concentration, at saturation, in water under 1 atm NO pressure at 25° C. is about 1.75 mM. Applying Henry's law, we estimate that at 10 μM NO-concentration in an inflamed or cancerous tissue volume element, the partial pressure of NO can be as high as about 0.006 atm. Its gas phase diffusion coefficient, D_{c}, at body temperature in air at 1 atm, is about 0.2 cm² sec⁻¹. Because the characteristic diffusion distance, L_{c} is obtained by solving the equation 2D_{c}τ_{c}=L_{c}², where τ_{c} is the characteristic diffusion time, NO diffuses in stagnant air in 10 minutes about 10 cm, in 1 hour about 40 cm, in 10 hours about 1.2 meters and in a day about 2 m. NO diffuses rapidly and passes practically unimpeded through biological membranes, including membranes of living cells. In a physiological buffer solution, under physiological conditions, its diffusion coefficient is of about 1.5×10⁻⁵ cm² sec⁻¹. Unless its lifetime is shortened, its diffusion length, which is the distance across which it diffuses during its half-life, usually exceeds hundreds of microns and can reach millimeters.

Table 1 relates the approximate equilibrium NO gas phase concentrations with those in water at 25° C.

TABLE 1
The relationship between the approximate equilibrium NO gas
phase concentrations and those in water at 25° C.

| Solution | | Approx. Pressure | | | |
|---|---|---|---|---|---|
| Concentration | | of NO Above the | | | |
| of NO | | Solution | | ppb | ppm |
| 1.7 | mM | 1 | atm | 1,000,000,000 | 1,000,000 |
| 0.85 | mM | 0.5 | atm | 500,000,000 | 500,000 |
| 85 | μM | 0.05 | atm | 50,000,000 | 50,000 |
| 8.5 | μM | 0.005 | atm | 5,000,000 | 5,000 |
| 0.85 | μM | 0.0005 | atm | 500,000 | 500 |
| 85 | nM | 0.00005 | atm | 50,000 | 50 |
| 8.5 | nM | 0.000005 | atm | 5,000 | 5 |
| 0.85 | nM | 0.0000005 | atm | 500 | 0.5 |

Although NO has an unpaired electron and is a free radical, its half life, in absence of a catalyst of its oxidation by molecular oxygen, is very long. At very high concentrations and in the presence of molecular oxygen its half live decreases because of the ter-molecular reaction 2NO+O₂→2NO₂. In tissues where the concentration of heme proteins, or heme compounds, is high, exemplified by blood, the half life of NO is shortened and its concentration is low, because iron in heme proteins coordinates NO and catalyzes its oxidation.

Diagnostic Medical Systems and Laboratory Methods of Monitoring of NO

1) In the Bowel

J. O. Lundberg et al. Nature Clinical Reviews in Gatroenterology and Hepatology, 2005, 2 (2) 96-102 (and in references therein) detect inflammatory bowel disease by measuring elevated NO concentrations in the luminal gas. A rectally inserted air-filled balloon is equilibrated with the luminal gas, withdrawn and the gas is assayed by the chemiluminescent NO₂ producing reaction of NO with ozone, O₃.

K. Alving et al., U.S. Pat. No. 6,063,027, describe diagnosing inflammatory conditions in the intestines by measuring the luminal NO concentration by obtaining a gas sample from the lumen of the intestines, preferably the colon or the emptied rectum, measuring the level of NO in the sample, comparing the measured level with the expected level for a healthy human or with a prior level measured in the human; and diagnosing the presence or absence of an inflammatory condition using the results of the comparison.

K. Alving et al., U.S. Pat. No. 6,183,416, describe diagnosing inflammatory conditions in the intestines and food intolerance by measuring the luminal NO concentration, preferably the rectal NO concentration, by obtaining a gas sample from the lumen of the intestines and measuring the level of NO in the obtained gas sample.

K. Alving et al., U.S. Pat. No. 6,511,425, describe diagnosing food intolerance, e.g. coeliac disease, by taking a gas sample from the lumen of the distal gastrointestinal tract, preferably the rectum, and measuring in the gas sample taken the NO concentration, after subjecting the patient to the suspected substance, underlying the intolerance reaction.

2) In the Respiratory System

Silkoff (U.S. Pat. No. 5,795,787 “Method and apparatus for the measurement of exhaled nitric oxide in humans”) and McClean (U.S. Pat. No. 6,010,459 “Method and apparatus for the measurement of components of exhaled breath in humans”) describe diagnosing disease by measuring the NO-concentration in the exhaled gas.

K. Alving et al., U.S. Pat. No. 6,019,100, describe a ventilator used in the monitoring of the respiratory NO-concentration, restoring the normal low-dose flushing of the lower airways with air from the upper airways by aspiration of air from the upper airways, and introducing this air in the inspiratory airflow of the ventilator. Gas is collected from the upper airways of the intubated or tracheostomized patient and introduced into the inspiratory airflow of a ventilator; introducing the collected gases and inspiratory airflow to the patient; and collecting the gases by connecting the nasal airways of the patient to an aspirating device.

K. Alving et al., U.S. Pat. No. 6,308,703 describe a related ventilator used for restoring the normal low-dose flushing of the lower airways with air containing NO from the upper airways by aspiration of air from the upper airways and introducing the aspired air in the inspiratory airflow of a ventilator. The method reduces the risk associated with administration of exogenous NO. An endotracheal tube is inserted into the intubated or tracheostomized patient's trachea, thereby physically separating the upper airways from the lower airways, the upper airways comprising all airways above the patient's vocal cords, and the lower airways comprising all airways below the patient's vocal cords, connecting the endotracheal tube to a ventilator and transferring the NO-containing gas from the upper airways to the lower airways.

J. Lundberg and E. Weitzberg, U.S. Application 20050143673, describe diagnosis of disease of the upper airways with a system increasing nasal NO release by an oscillating air-flow.

K. Alving and J. Lundberg U.S. Pat. No. 6,626,844 describe inhibiting, by the application of an anti-bacterial and/or pH increasing composition, NO production in the oral cavity to avoid the disturbing influence of orally produced NO in the measuring of exhaled NO.

K. Alving et al., U.S. Pat. No. 6,723,056, describe a device for the collection, storage and/or transport of gas samples. Exhaled NO-containing air is collected in a bag comprising an inlet/outlet and a reagent chamber. This makes possible the storage and transport of the collected air sample and thus enables efficient and repeatable off-line determinations of NO in the bag.

J. R. Mault U.S. Pat. No. 6,612,306 and U.S. Pat. No. 6,620,106 and U.S. Patent Applications 20020026937 and 20020077765 describe respiratory NO meters with an indirect calorimetry system, including transducers sensitive to expired airflow. They also describe the meter, including a respiratory fluorescence gas sensor, having a radiation emitter for directing radiation along the flow path and a radiation detector for detecting fluorescence from the respiratory gas induced by the radiation. The respiratory gas sensor also includes a narrow band filter disposed between the detector and the gas, to pass fluorescence to the radiation detector, so as to rapidly detect components of the respiratory gas passing through the flow path; or including a sensor detecting adsorbed NO through change in resonance frequency of a micromechanical structure.

P. von Bahr et al. U.S. Application 20040082872 describe measuring NO in exhaled air electrochemically.

3) In the Urogenital Tract

K. Alving et al., U.S. Pat. No. 6,149,606 describes diagnosing inflammatory states by collecting endogenous NO in the urogenital tract by positioning an NO permeable, liquid impermeable, inflatable balloon in the urethra surrounded by the prostate gland, and using a second balloon in the bladder to seal off the bladder from the urethra and for positioning the first balloon.

4) In the Breast

M. Anbar, U.S. Pat. No. 6,035,225 “Detection of cancerous lesions by measuring nitric oxide concentrations in tissue”, describes retrieving fluid and measuring its NO concentration.

5) For Diagnosing and Predicting Pre-Term Labor

R. K. Riemer, U.S. Pat. No. 6,210,918, describes assaying NO in the in blood, urine, saliva or in other tissue samples.

6) In Septic Shock

C-S Lai, U.S. Pat. No. 5,358,703, describes detecting NO in an aqueous body fluid by forming a water-soluble, stable, paramagnetic complex with NO and detecting the complex by magnetic resonance spectroscopy.

Methods for Assaying Nitric Oxide

Nitric oxide is a recognized air pollutant and its monitoring is practiced by many companies, researchers and inventors who developed a variety of monitoring tools. The most important of these are monitoring by chemiluminescence of excited NO₂.

1) Chemiluminescence of excited NO₂

NO₂ is generated in a photon emissive excited state in the reaction of NO with ozone (O₃) and the reaction can be followed by monitoring the chemiluminescence This accurate and sensitive method requires an expensive system and is most often used to monitor air quality. It was used by J. Lundberg et al., as discussed above for diagnosis of respiratory and bowel inflammation. It is also used in medical diagnostic products of Aerocrine AB, Sweden for diagnosing and monitoring airway inflammation, particularly asthma, see website http://www.aerocrine.com/us/products.html and in products of Eco Physics AG, Switzerland, see website http://www.ecomedics.com.

Chemiluminescence NO analyzers measure the NO concentration by routing the sample gas to a reaction chamber, where the NO combines with ozone (O₃), produced in a separate reactor, and metered into the reaction chamber. In the reaction between NO and O₃, NO₂ and O₂ are formed. About ⅕^{th} of the NO₂ is formed, when the pressure in the chamber is low enough, in the excited, mostly infrared light emitting (λₘₐₓ 1.2 μm) state. When the ozone in the reaction chamber is in excess, the emitted infrared photon flux can be related to the NO concentration.

2) Mass Spectroscopy

Another useful tool is quantitative mass spectroscopy, which requires use of a mass spectrometer, the typical cost of which exceeds $ 10,000.

3) Change in Fluorescence Upon Reaction with NO, Usually in the Presence of Oxygen, Involving the Formation of Intensely Fluorescent Triazoles

This method has been used to monitor NO in tissues. It is well known that the quantum yield of fluorescence of aromatic and heterocyclic vicinal diamines increases drastically, because their reaction with NO and oxidation yields intensely fluorescent triazoles.

T. Naito, Univ. Yakugaku Zasshi, 1947, 67, 141-3 showed that the vicinal diamine 3,4-diaminoquinoline reacted with HNO₂ to yield a triazole.

J. Dobas et al., Chemicke Listy pro Vedu a Prumysl, 1957, 51 1103-12 synthesized, by nitrosating o-diamines, a series of fluorescent triazole dyes.

L. J. Dombrowski, and E. J. Pratt, Analytical Chemistry, 1972, 44(14), 2268-72 developed a sensitive triazole-formation based fluorometric method for measuring NO₂⁻ and determined nanogram quantities of NO₂⁻.

F. Brew and S. Forsythe Letters in Applied Microbiology 1990, 10(1), 39-42 showed that gastric isolates of the pathogen Neisseria subflava nitrosated the vicinal diamine 2,3-diaminonaphthalene to a fluorescent triazole product.

T. Misko et al. Analytical Biochemistry, 1993, 214(1), 11-16. described a rapid and sensitive fluorometric assay for quantification of nitrite and nitrate is based upon the reaction of nitrite with 2,3-diaminonaphthalene to form the fluorescent product, 1-(H)-naphthotriazole, detecting 10 nM nitrite.

G. Gabor and N. Allon described a spectrofluorometric method for NO determination and a remote NO detector employing a fiber-optic sensor (Analytical Biochemistry, 1994, 220:16-19).

M. W. Owens et al., Free Radical Research 1995, 23(4), 371-8 showed that stimulation of pleural mesothelial cells (PMC) with proinflammatory cytokines promoted the NO caused N-nitrosation of 2,3-diamino-naphthalene, producing fluorescent 1-naphtho-2,3-triazole. They proposed that fluorescent triazole formation resulted of L-arginine-dependent formation of NO.

A. M. Miles, et al., Methods (San Diego), 1995, 7(1), 40-7 determined NO fluorometrically by N-nitrosation of 2,3-diaminonaphthalene (DAN) to yield the highly fluorescent 2,3-naphthotriazole, detecting 10-30 nM of NO.

P. J. Andrew, FEBS Letters, 1997, 408(3), 319-323 quantified the NO release from LPS and IFN γ-stimulated murine macrophages and iNOS transfected hamster cells by NO caused N-nitrosation of the vicinal diamine 2,3-diamino-naphthalene, producing fluorescent 1-naphtho-2,3-triazole.

P. Heiduschka and S. Thanos, Neuroreport, 1998, 9(18), 4051-7 reacted non-fluorescent vicinal diamine 1,2-diamino-anthraquinone (DAA) in the eyes of rats with NO, producing in the eye the fluorescent triazole.

H. Kojima et al., Analytical chemistry, 1998, 70(13), 2446-53 synthesized diaminofluoresceins (DAFs) and used them as fluorescent indicators for NO. They showed that the fluorescent chemical transformation of DAFs involved N-nitrosation of the aromatic vicinal diamines, and that in the presence of dioxygen the green-fluorescent triazole was formed. They detected dissolved NO at 5 nM concentration and imaged in NO production in living cells.

Researchers also used compounds made fluorescent through reaction with NO. Such compounds were disclosed by C.-S. Lai, U.S. Pat. No. 5,885,842 and U.S. Pat. No. 6,306,609, who used 2,3-diaminonaphthalene (DAN), a non-fluorescent vicinal diamine, reacting with NO to form 2,3-naphthotriazole, a fluorophore. The reaction requires the presence of an oxidant, like oxygen, to form the intensely fluorescent compound. Lai detected in liquids sub-micromolar concentrations of NO.

T. Nagano and H. Kojima JP 95-189978 19950726, JP 09043153 A2 19970214 Heisei, U.S. Pat. No. 5,874,590, U.S. Pat. No. 6,441,197, U.S. Pat. No. 6,569,892, U.S. Pat. No. 6,833,386 describe relatively non-fluorescent vicinal-diamines, such as diaminofluorescein and its derivatives, which react with NO in the presence of an oxidant like oxygen to form intensely fluorescent compounds. They also describe longer wavelength emitting and less pH sensitive NO-activated diaminorhodamine derivatives JP 97-177097 19970702, WO 9901447 A119990114, WO 98-JP2924 19980630, U.S. Pat. No. 6,201,134, U.S. Pat. No. 6,469,051, U.S. Pat. No. 6,756,231.

4) Fluorescence-Based NO Sensors not Involving the Formation of Triazoles

B. R. Soller U.S. Pat. No. 5,582,170 described a fiber optic sensor for measurement of in vivo nitric oxide concentration. Their sensor contains an NO-sensing compound in a polymer matrix attached to an optical fiber. The sensor may be placed in a blood vessel, including one within the heart of a subject for continuous measurement of nitric oxide concentrations in blood. The fiber optic sensor provides high resolution NO measurements in solid or liquid containing biological tissues and within living cells.

R. Kopelman et al. U.S. Pat. No. 6,002,817, U.S. Pat. No. 6,272,262, U.S. Pat. No. 6,636,652, and U.S. Pat. No. 6,900,891 described fluorescence ratio and fluorescence monitoring fiber-optic sensors and optical fiber-less sensors utilizing metals, and more particularly metal colloids comprising NO-binding compounds with little or no interference from other analytes, based on heme-binding protein fluorescers. Their metallic colloidal particles constituting the fiberless sensor are small enough to enter non-invasively a single mammalian cell.

M. G. Bawendi et al., Biological applications of quantum dots U.S. Pat. No. 6,306,610, U.S. Pat. No. 6,326,144, U.S. Pat. No. 6,855,551, disclose tunable fluorescent semiconductor nanocrystals associated with a molecule or reagent for detection of biological compounds such as enzymes, enzyme substrates, enzyme inhibitors, cellular organelles, lipids, phospholipids, fatty acids, sterols, cell membranes, molecules involved in signal transduction, receptors and ion channels that can also be used to detect nitric oxide.

E. W. Adams et al. “Surface-modified semiconductive and metallic nanoparticles having enhanced dispersibility in aqueous media: U.S. Pat. No. 6,649,138, disclose that nanoparticle conjugates comprising a surface-modified semiconductive nanoparticle can be used to detect nitric oxide.

S. J. Lippard and S. Hilderbrand U.S. Patent Application 20030068275 described metal complexes bound to fluorophores, detecting NO through an increase in fluorescence upon their coordinating NO.

5) Compounds Changing Their Absorption Spectrum Upon Reaction with NO

The absorption spectra of numerous compounds, known as NO-scavengers, change upon their reaction with NO. Commercially available examples of these include those from Axxora LLC, San Diego, Calif. the US distributor of Alexis Corp., and found on the website: http://www.alexis-corp.com/nitric_oxide_scavengers/opfa.568.2.1.0.html.

The website lists ACP, L-(+/−)-Alliin, L(+)Alliin, 4-Amino-2,2,6,6-tetramethylpiperidine-1-oxyl, free radical, Carboxy-PTIO, 3-Carboxy-2,2,5,5-tetramethyl-1-pyrrolidine-1-oxyl, free radical, CDMIO.potassium salt, Cepharanthine (98%), CMH.hydrochloride, CPH.hydrochloride, DEPMPO, Diethyldithiocarbamic acid.sodium salt.trihydrate (>99%), 1,1-Diphenyl-2-picryl-hydrazyl, free radical, DIPPMPO, DMPIO, DMPO, DMPO (high purity), EMPO, Galvinoxyl, free radical, MCPIO, Methylene blue.trihydrate, MGD.sodium salt.monohydrate, PBN; N-t-Butyl-α-phenylnitrone, POBN (high purity), PP-H, PTIO, RSSR, Rutin.trihydrate, (O)-Sulfinpyrazone, TEMPOL; 4-Hydroxy-TEMPO, TEMPONE, TEMPONE-H.hydrochloride, TMIO; 2,2,4-Trimethyl-2H-imidazole-1-oxide, TMPO; 3,3,5,5-Tetramethyl-pyrroline-N-oxide, TOAC, Trimethylammonio-PTIO.

In some, like PTIO and its derivatives, undergoing a blue to colorless change and selectively reacting with NO, the spectral change is easy to see.

6) Electrochemical NO Detectors

K. Shibuki described an electrochemical microprobe with which NO was detected in brain tissue (Neurosci. Res. 1990, 9:69-76).

T. Malinski et al. (Nature, 1992 358:676-678) used a porphyrin-based electrochemical microsensor to observe in-situ NO-release from a single cell. T. Malinski et al. U.S. Pat. No. 5,603,820 also described a microelectrode for specific and quantitative measurement of NO-based on its catalytic oxidation. The microsensor, operating in the amperometric, voltammetric or coulometric mode in two or three electrode systems, responds linearly up to about 300 μM NO, has a response time faster than 10 msec, and has a detection limit of about 10 nM.

B. W. Allen et al. U.S. Pat. No. 5,980,705, U.S. Pat. No. 6,280,604 and U.S. Pat. No. 6,287,452 described NO-specific electrodes for in situ detection of NO in biomedical applications, having a surface region, particularly of ruthenium or an oxide of ruthenium, capable of forming complexes with NO.

J. R. Saffell and D. H. Dawson U.S. Patent Application 20020121438 described an electrochemical gas sensor comprising a wick providing a path for an electrolyte to pass from a reservoir for electrolytic continuity between the counter electrode and the working electrode.

The Persisting Need and Purpose of this Invention

Although NO analyzing diagnostic methods have been available, some for many years, the known methods required expensive instrumentation, required trained professionals to operate the instruments, involved systems that are much too heavy and too large to be carried by the patient or the physician, and were, as is evident from the literature cited, far from simple to use. When a physician needed information about elevation of the NO concentration, (s)he usually avoided assays of NO and asked instead for assays of the two NO-oxidation products, nitrite, NO₂⁻ and/or nitrate NO₃⁻ in blood or in urine. These samples were obtained by having the patient visit a laboratory, or were sent to a laboratory from the physician or her/his staff office. This delayed receipt of the information and the method provided little or no information about the tissue, organ or specific volume element of the body in which the NO-concentration was elevated.

Thus, simple, low cost, easy to use methods, and simple, optionally hand-held, tools for rapid, low cost, and easy in-situ diagnosis of elevated NO-concentration or other chemical compound concentration are desired for use in, for example, gas-containing volume elements of the human body and on the skin. Examples of gas-filled volume elements include those in the digestive tract (between the mouth and the rectum), the female reproductive system (particularly the vagina, the cervix and the uterus), the respiratory system, the ear, and the nose, to name a few. At least some of these objectives will be met by the aspects of the present invention.

### BRIEF SUMMARY OF THE INVENTION

Methods and devices are provided for evaluating the presence of disease in a patient. In particular, methods and devices provided for screening patients for neoplastic and inflammatory disease. Such diseases are often indicated by the elevated level of a chemical compound associated with disease, particularly nitric oxide (NO) and/or nitrogen dioxide (NO₂). Through measuring and/or estimating the chemical compound-concentration, the methods and tools provided distinguish between patients who require further testing and/or treatment and those who do not. The methods and tools also provide information about the effectiveness of treatment, such as treatment to reduce inflammation or control of the growth of malignant tumors. These methods and devices are relatively inexpensive, easy to use, and provide many advantages which are described herein.

The methods and devices of the present invention are provided for evaluating the presence of disease in a suspected tissue of a patient, such as by measuring elevated predetermined chemical compound concentrations on, near or within body tissues. In particular, by measuring concentrations of chemical compounds comprised of nitrogen and oxygen, usually NO and/or NO₂. Such devices comprise a reactive material and support structure. The reactive material reacts with the chemical compound, indicating a concentration level by a spectral change. A spectral change may be described as a change in spectrum, such as an intensity change, such as a change in absorbance or reflectance, or quantum yield of luminescence, or luminescence intensity, or a change in absorbed, reflected or emitted wavelengths, optionally seen by the eye, or a luminescence wavelength and/or intensity and/or decay time change. The spectral change preferably includes a change in color and/or a change in fluorescence intensity. In some embodiments, different reactive materials are present, each indicating concentration levels by a different type of spectral change.

The reactive material is supported by, such as mounted on, attached to, coupled with, joined with or incorporated within, the support structure. A variety of support structures are provided, including probes, beads, sheets, cords, tethered bodies, plugs or capsules, to name a few. It may be appreciated that descriptions involving NO and/or NO₂, are also applicable to other suitable chemical compounds. Likewise, descriptions involving a reactive dye and/or an NO-reactive dye are also applicable to other suitable reactive materials. Further, descriptions involving a probe are also applicable to other support structures. Such terminology is illustrative and not intended to limit the scope of the present invention.

Probes are configured for insertion in and retrieval from an orifice of the body. Such probes are typically hand-held and the reactive material is disposed at or near a portion which is inserted within or through the orifice. Typically, the probed orifice of the body leads to a fluid-containing, preferably a gas-containing, volume element within which NO and/or NO₂ concentration is desired to be measured. For example, when probing NO in the luminal gas of the bowel, particularly the luminal gas in the colon or rectum, the orifice in which the probe is inserted is the anus. Such a probe may have a form similar to a rectal thermometer. When probing NO in the upper respiratory tract, the mouth, the esophagus or the stomach, the orifice in which the probe is inserted is the mouth. Such a probe may have a form similar to an oral thermometer. Alternatively, when probing the stomach, a capsule may be used. The capsule has a string or wire which is attached and is held while the capsule is swallowed by the patient to allow retrieval from the stomach. When probing NO in the in the vagina, cervix, or uterus, such a probe may have a form similar to a vaginal thermometer which is insertable in the vagina. In other embodiments, a body orifice is probed to measure NO concentration at a location within the orifice. For example, to probe for periodontal disease, a probe is inserted between the teeth. Such a probe may have a form similar to a toothpick.

Beads are configured for passage through the body. For example, in some embodiments, a bead including reactive material is swallowable by a patient and recoverable from the patient's feces or from her/his mouth. Such beads are used to analyze part or all of the digestive tract. In some embodiments, the beads are magnetic so that the beads may be easily retrieved from the feces with a magnet. Alternatively, using a magnet, the swallowed beads may be guided back to the mouth.

Sheets are configured for measuring NO concentration on or emanating from a surface of a tissue, such as skin. Here, the sheet comprises paper, cloth, plastic or other suitable material which is applied to the tissue surface. The sheet may also include adhesive to adhere the sheet to the tissue surface. Such sheets may be used to test for disease of the skin. It may be appreciated that such sheets may be applied to any tissue surface, such as luminal surfaces of the body.

Tethered bodies are configured for measuring NO and/or NO₂ concentration within a body lumen or cavity wherein the body is retrievable by use of the tether. Examples of body lumens include a vagina, a rectum, an ear, or a nose. Examples of body cavities include a stomach or a bladder. Tethered bodies positionable within the vagina may resemble a feminine tampon. Tethered bodies positionable with the rectum may resemble a suppository. When probing the ear, the body may resemble an earplug. The tethered body includes a retrieving element thereattached, wherein the body is configured so that the retrieving element remains outside the body while the body is positioned within the stomach, rectum, vagina, ear, or nose, for example.

When the reactive material is exposed at the site to be tested, the device measures and transmits the local NO concentration while at the site, or the device is removed after a pre-defined period of time, rinsed and is visually read, optionally using a calibration strip, or is instrumentally read, for example with a one or multi-wavelength absorption or emission monitor, usually comprising a light source, a detector, and optionally, one or more filters, such as a reflectometer or fluorometer. Optionally, phase sensitive detection is employed.

The results of the measurements may be used to screen the patient for further testing, detect the presence of a current disease, diagnose a disease, monitor treatment of a disease, or other clinical usages.

Other objects and advantages of the present invention will become apparent from the detailed description to follow, together with the accompanying drawings.

### BRIEF DESCRIPTION OF THE DRAWINGS

FIGS. 1-3 illustrates embodiments of a device comprising a probe and at least one reactive material.

FIG. 4 illustrates a probe as in FIG. 1 inserted in an anus.

FIGS. 5A-5C illustrate embodiments of support structures inserted between two teeth.

FIGS. 6A-6C illustrate embodiments of a device comprising a tethered body and at least one reactive material.

FIG. 7 provides a cross-sectional illustration of a bead having a core of a magnetic material.

FIG. 8 illustrates an embodiment of a sheet with a reactive material therein.

FIG. 9 illustrates an embodiment of a covering having the form of an external sleeve.

FIG. 10 illustrates an embodiment of a covering having the form of an external sheet.

FIG. 11 illustrates a device having an inner sleeve with a reactive material and an external outer sleeve.

### DETAILED DESCRIPTION OF THE INVENTION

Terms and Definitions

The following terms and definitions apply to at least some of the embodiments of the present invention:

Calibration or reference strip: a strip showing the change in the spectrum for different exposures. The preferred strip shows the changes in the visible part of the spectrum. A most preferred strip shows the exposure dependence of the spectrum suited for reading by the naked eye.

Capsule: A body, usually contacting the fluid of the stomach. The capsule optionally comprises a cylindrical part and has an attached string, cord, wire or other means that enables its retrieving. The capsule is swallowable by the user.

Cellulosic material: a cellulose-containing materials or a material containing a material derived of cellulose. Examples of cellulose containing materials include paper and cotton. Examples of cellulose-derived materials include methylcellulose, ethylcellulose, hydroxyethyl cellulose, rayon, acetylated cellulose.

Ceramic: a non-toxic material, consisting mostly of one or more crystalline and/or vitreous oxide or oxides, usually oxides of one or more metallic and/or semi-conducting element or elements, such as silicon, sodium, aluminum, magnesium, calcium, magnesium, titanium, zirconium, lithium.

Cord: a strong, elongated flexible object with a length to diameter ratio of at least 30, preferably at least 200 and most preferably at least about 1000. The cord can be a monofilament or it can be multi-filamentary. It can be made of a plastic and/or a composite. An exemplary composite cord would have a metal or carbon fiber comprising core, or multiple metal or carbon wires, or glass fibers running in parallel along the long direction of the cord.

Detector: a device converting a photon flux to an electrical signal, such as a photodiode, a diode array, a photoresistor, or a photomultiplier.

Diagnosis and the related terms like diagnosed or diagnosing: in addition to their usual meaning of confirming or refuting the existence of a disease in a patient, in an organ, or in a particular tissue, also the following of the progress of a disease, the following of the effect of treating a disease, and/or the determination of the extent or severity of a disease.

Disease: generally, inflammation and/or neoplasia.

Display: a device for visualization of electrical signals. Examples of displays are liquid crystal displays, light emitting diode displays, plasma displays.

Dye: a type of reactive material.

Exposure: exposure to NO resulting in a spectral or luminescence change. The exposure increases linearly or non-linearly, preferably linearly, with the NO-concentration in the volume element of the fluid containing the device, and also increases linearly or non-linearly, preferably linearly, with the residence time of the device in the monitored volume element. The exposure is usually proportional to the integral ∫c(t)dt where c(t) is the time dependent NO-concentration seen by the device between t=tₛₜₐᵣₜ and t=t_{end}, where tₛₜₐᵣₜ is the point in time where the NO-reactive material-containing device is inserted in the monitored volume element of the fluid and t_{end} is the point in time when it is withdrawn from this volume element.

Filter: a device allowing the selection of photons of a wavelength domain in preference over photons of another wavelength domain, optionally, but not necessarily, in combination with a slit. Examples of filters include color and dichroic filters, dichroic mirrors, gratings, prisms.

Fluid: a gas, for example air and/or methane, or a liquid, such as liquid in the stomach, or in the gut.

Fluorometer: an instrument capable of measuring a change in the luminescence intensity and/or spectrum and/or decay time and/or the luminescence excitation spectrum. An exemplary simple fluorometer comprises one or more light sources and one or more detectors, and optionally one or more filters. Also optionally, phase sensitive detection is employed. A preferred fluorometer is suitable for reading the change in the luminescence intensity and/or spectrum and/or decay time and/or the luminescence excitation spectrum when NO-reactive dye containing device is inserted in the fluorometer.

Gas: a mixture, comprising mostly one or more of the following: nitrogen, oxygen, carbon dioxide, water vapor, methane.

Handle: the non-inserted part of a probe, designed to be conveniently held in the hand and facilitating the insertion of the probe in the orifice and/or the removal of the probe from the orifice. The handle may optionally house electronic and optical components.

Inflammation: a volume element in the human body or in the body of an animal comprising more NO-generating white blood cells, most commonly neutrophils and/or macrophages, than the same tissue, if healthy.

In-situ: while inserted in the body.

Lightguide: a photon-channeling device having at least one an inner layer with a higher index of refraction, termed the core and at least one outer layer, termed the cladding having a lower index. While other claddings are usually preferred because they lessen the effects of dust particles, ambient air can serve as a cladding. Though clad optical polymeric and/or glass fibers are the most widely used lightguides, coated plastic rods, such as those of the NO-probes of this disclosure are also effective lightguides, for example when coated with a lower index film, such as a silicone film, the approximate index of refraction at wavelengths near 590 nm is about 1.40, lower than that of the exemplary below listed non-crystalline polymers.

Light source: a source of photons, usually produced by the conversion of electrical power to a photon flux, such as a light emitting diode, a laser diode, a gas or solid or liquid laser, an incandescent lamp or halogen lamp, or a high, medium or low pressure arc lamp.

Light transmissive material: a non-crystalline polymer, or ceramic, or polymer-ceramic hybrid. Examples of non non-crystalline polymers include polyacrylates, such as poly(methyl methacrylate), n≈1.49 or poly(hydroxyethyl methacrylate), n≈1.51; or poly(naphtyl methacrylate), n≈1.64); cellulosics, like cellulose acetate, n≈1.48; polycarbonates like poly (diethylene glycol diallyl bicarbonate), n≈1.50 and poly(arylcarbonate), n≈1.50; polystyrene, n≈1.59, the provided values of n being their approximate indices of refraction for wavelengths near 590 nm, of importance when the polymers are used in lightguides. Examples of non-crystalline ceramics include silicate glasses, aluminosilicate and borosilicate glasses, vitreous quartz.

Luminescence: emission of photons by the excited reaction product of the NO-reactive material. It can be fluorescence or phosphorescence. The luminescence of the reaction products of the NO-reactive materials is usually fluorescence. A change in luminescence can be a change in the excitation and/or the emission spectrum, and/or a change in the quantum yield, and/or a change in lifetime of the excited photon emitting molecule and/or ion, and/or a change in the intensity of the emission detected by an instrument and/or seen by the eye. The NO-reactive luminescent dye changes, upon its reaction with NO, one or more of these characteristics. Increased luminescence or fluorescence intensity and increased quantum yield have the same meaning.

Magnetic bead: an object without sharp edges or corners having a core of a magnetic metal and/or a magnetic metal oxide, coated with an NO-reactive material, such as dye, containing plastic, or ceramic, or composite. The envelope is preferably a plastic, and is most preferably an elastomer, such as a rubbery poly (dimethyl siloxane). The shape of the bead can be spherical, ellipsoidal or other. The bead is typically larger than about 0.01 cm in its smallest dimension, and is typically smaller than about 2 cm in its largest dimension. It is preferably larger than about 0.1 cm its smallest dimension and smaller than about 0.5 cm in its largest dimension. When in a mixture, the magnetic bead can be separated and/or collected with a magnet or electromagnet.

Neoplasia: a benign tumor, pre-malignant tumor, or malignant tumor. It includes, but is not limited to, adenomas, such as polyps, carcinomas and sarcomas.

Phase sensitive detection: the preferred detection of a purposely produced train of photonic signals over a photonic signal that was not purposely produced, usually originating in ambient light from the sun or from indoor lighting devices. For example, the temporal distribution of photons emitted by the light source may have, or may be tailored to have, for example by a light chopper, piezoelectric device or oscillating mirror, a temporal distribution related to the function defining the output of the detector.

Plastic or polymer: a man-made, or a natural, preferably non-toxic and/or non-allergenic material, comprised mostly of a polymer, of a molecular weight of at least 1,000 Da, preferably at least about 10,000 Da and most preferably at least about 100,000 Da, the majority atoms of which are atoms selected from the group carbon, silicon, hydrogen, oxygen, chlorine, fluorine, bromine, nitrogen, sulfur. The plastic can be a thermoplastic polymer, or an elastomer. An elastomer, or a mostly amorphous thermoplastic polymer, is usually preferred. Plastic substrate refers to such plastics formed into a solid-phase shape that can be exposed to and separated from a sample, usually a liquid sample. Suitable shapes are solid and preferably have conventional geometries, such as rods, tubes, strips, dipsticks, beads, and the like. The plastic may or may not be man-made. It can be, for example, cellulosic.

Reactive material or NO reactive material: material absorbing and/or emitting light in part of the spectral or luminescence range between about 300 nm and about 1700 nm, preferably absorbing or emitting in the visible range, between about 400 nm and about 700 nm, and most preferably absorbing in the visible range. The NO material reacts with NO and/or NO₂. Preferably it reacts only with NO. Upon reacting with NO and/or NO₂, the reactive material undergoes a spectral and/or luminescence change, which can be a change in the absorption spectrum, in the reflected spectrum, and/or in the luminescence excitation spectrum, and/or in the emitted luminescence spectrum. The change can be a change in intensity, for example an increase or decrease in the absorbance, reflectance, or quantum yield of luminescence, or it can be a change in the wavelengths absorbed, reflected or emitted, or it can be both a change in intensity and in wavelengths. Preferably, the change is in the visible, and most preferably the reflectance and/or reflected spectrum is visibly changed. Fluorescence means luminescence with a decay time shorter than about 1 msec. Phosphorescence means luminescence with a decay time longer than about 1 msec. Decay time means the time required for the luminescence intensity to drop, after excitation, to 1/e or about 1/2.713 of its initial value.

Reflectometer: an instrument for measuring the reflectance and/or the reflection spectrum. An exemplary simple reflectometer comprises one or more light sources, such as an incandescent bulb, and/or light emitting diode or multiple diodes, or one or more lasers; it often comprises one or more filters, which may selectively absorb and/or transmit and/or reflect part of the spectrum; and one or more photon detectors, such as photodiodes, or photoresistors. A preferred reflectometer is suitable for reading the change in reflectance and/or reflection spectrum when the NO-reactive material containing device is inserted in the reflectometer.

Retrieving element: an elongated body, such as a string, made of a polymer, metal wire, or composite, attached to an orifice-inserted body, at least part of which is not inserted in the orifice, facilitating the retrieving of the inserted body.

RF: microwave or radio frequency.

Spectrum: absorption, and/or reflection, and/or luminescence spectrum of wavelengths typically longer than about 300 nm and typically shorter than about 1,700 nm. A change in the spectrum, also termed the spectral change, can be an intensity change, such as a change in absorbance, or reflectance, or quantum yield of luminescence, or luminescence intensity, or a change in absorbed, reflected or emitted wavelengths, optionally seen by the eye, or a luminescence wavelength and/or intensity and/or decay time change.

Support structure: a structure, assembly, substrate, catheter, probe, bead, tethered body, plug, capsule, sheet or other member suitable for supporting a reactive material in or on a body cavity or surface for practicing the methods described herein. The structure may comprise a tip and means for its retrieval, such as a handle or a string.

Suppository: a rectally inserted body, usually contacting the luminal gas filled part of the bowel. The suppository optionally comprises a cylindrical part and has an attached string, cord, wire or other means that enables its retrieving. Optionally, the suppository is self-inserted by the user.

Tampon: an elongated body inserted into the vagina.

Thermometer-like probe: an elongated body, optionally made of a plastic and/or a ceramic, also having a tip suitable for insertion in a body-orifice, the tip being optionally narrower than the main body, which is not inserted in the orifice. The tip and/or the main body are optionally cylindrical.

Tip: part of a probe partially or completely insertable in the orifice of the body. When partially inserted, the inserted part comprises some or all the NO-reactive material.

Reactive Materials

A variety of reactive materials may be used. In some embodiments, one or more nitric oxide scavengers are used, such as imidazolineoxyl N-oxides. The absorption spectra and/or the luminescence spectra of NO-scavengers change upon their reaction with NO. Commercially available examples which may be used include those from Axxora LLC, San Diego, Calif. the US distributor of Alexis Corp., and found on the website: http://www.alexis-corp.com/nitric_oxide_scavengers/opfa.568.2.1.0.html. The website lists ACP, L-(+/−)-Alliin, L(+)Alliin, 4-Amino-2,2,6,6-tetramethylpiperidine-1-oxyl, free radical, Carboxy-PTIO, 3-Carboxy-2,2,5,5-tetramethyl-1-pyrrolidine-1-oxyl, free radical, CDMIO.potassium salt, Cepharanthine (98%), CMH.hydrochloride, CPH.hydrochloride, DEPMPO, Diethyldithiocarbamic acid.sodium salt.trihydrate (>99%), 1,1-Diphenyl-2-picryl-hydrazyl, free radical, DIPPMPO, DMPIO, DMPO, DMPO (high purity), EMPO, Galvinoxyl, free radical, MCPIO, Methylene blue.trihydrate, MGD.sodium salt.monohydrate, PBN; N-t-Butyl-α-phenylnitrone, POBN (high purity), PP-H, PTIO, RSSR, Rutin.trihydrate, (±)-Sulfinpyrazone, TEMPOL; 4-Hydroxy-TEMPO, TEMPONE, TEMPONE-H.hydrochloride, TMIO; 2,2,4-Trimethyl-2H-imidazole-1-oxide, TMPO; 3,3,5,5-Tetramethyl-pyrroline-N-oxide, TOAC, Trimethylammonio-PTIO.

1) Irreversibly Reacting Dyes for Single Use Devices

Any stable radical or compound undergoing spectral change upon reacting with a predetermined chemical compound, such as NO and/or NO₂, can be used. Preferably, the chemical compound is in a gaseous state at 37° C. Alternatively or in addition, such a reaction is preferably in the presence of air. Examples of dyes undergoing an irreversible spectral change are provided above, where dyes changing their luminescence or absorption characteristics upon reacting with NO in air are listed. The listed examples of dyes include PTIO, 2-phenyl-4,4,5,5-tetramethylimidazoline-1-oxyl 3-oxide, available from Sigma-Aldrich, St Louis, Mich., changing its color from blue to colorless, a change readily seen by the naked eye. Other examples include triazole-forming vicinal diamines, the NO-reaction and oxidation, optionally by air or dissolved oxygen, converting the practically non-fluorescent vicinal diamines to strongly fluorescing triazoles. Examples of such reactions were listed above and include, for example, those of 2,3-diaminonaphthalene, or of 4, 5-diaminofluorescein (DAF-2 DA), available from Alexis Biochemicals, San Diego, Calif.

Other examples of non-fluorescent or less fluorescent reagents reacting with NO in air include the 1,4-diphenylnaphthalenes I, III and V, producing the more fluorescent oxadiazole II, thiadiazole IV, and triazole VI. I is an ortho-aminonaphthol, III is an ortho-aminothionaphthol and V is a vicinal diaminonaphthalene.

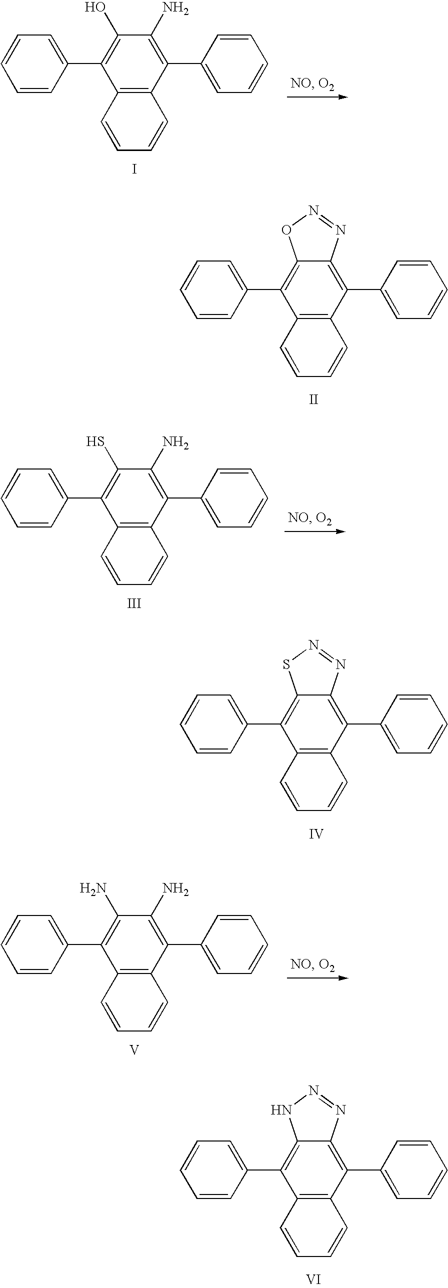

According to O. M. Busch et al., “Application of a New Color Detection Based Method for the Fast Parallel Screening of DeNOx Catalysts” Journal of the American Chemical Society, 2002, 124, 13527-13532, 2,2′-azinobis(3-ethylbenzthiazoline-6-sulfonic acid) (ABTS) forms a green color in the presence of NO, but is water-soluble. Compounds of the 2,2′-azinobis-(3-alkylbenzthiazoline) family, such as 2,2′-azinobis-(3-ethylbenzthiazoline), are also expected to similarly change color, but to have the advantage of being water insoluble, and to be, therefore, advantageously non-leachable.

2) Enhancement of the Detectivity of NO by Energy Transfer

Applying principles of energy transfer it is possible to reduce the concentration at which luminescent molecules can be detected and to shift the emitted wavelengths to more conveniently measured or seen spectral regions. In general, a shorter wavelength excited first molecule is dissolved in a matrix, and is excited, for example by UV light. It then transfers some of its excitation energy to a second molecule, which emits light of longer wavelengths, typically in the visible or near infrared. Usually the concentration of the first molecule is at least ten times higher than that of the second molecule. Because the excitation and emission spectra of fluorescent molecules often overlap, emitted photons are lost by re-absorption. Such loss is conveniently reduced in the energy transferring systems.

Furthermore, energy transferring systems can be based on available non-crystalline optically clear polymers, such as polystyrene or a poly(vinyl-toluene). Here the aromatic ring functions of the polymer itself are excited by light of wavelengths shorter than about 300 nm. They transfer part of their energy to a homogeneously dissolved first solute, such as p-terphenyl or 2,5-diphenyl-1,3,4-oxadiazole, dissolved at a concentration typically between about 1 weight % and about 5 weight % and the emitted light, at wavelength typically between about 300 nm and about 400 nm, is detected. An energy accepting fluorescent molecule, efficiently accepting energy from the first solute, is generated by the reaction of NO in the presence of O₂ from the reactive material, the molecule generated emitting light typically of wavelengths longer than about 400 nm. Exemplary reactive materials include Compounds I, III or V, and their exemplary fluorescent products include Compounds II, IV or VI. The typical concentration of the reactive material in the polymer is usually less than about 0.5 weight %, and is preferably less than about 0.05 weight %. In the test for NO, light of wavelengths shorter than about 400 nm, preferably shorter than about 300 nm, is used for excitation, and fluorescence is observed typically at wavelengths longer than about 400 nm.

2) Reversibly Reacting Dyes for Multiple Use Devices

Exemplary dyes, reacting with NO reversibly to form intensely fluorescent species were also described above. These dyes have the important advantage of being reversible, their fluorescence intensity scaling with the NO partial pressure, making them useful in multiple use devices. They include those containing dirhodium tetracarboxylate scaffold-comprising dyes described by S. J. Lippard and S. Hilderbrand in U.S. patent application 20030068275 and by Scott A. Hilderbrand, Mi Hee Lim and Stephen J. Lippard in Journal of the American Chemical Society, 126 (15) (2004): 4972-4978. They also include cobalt-containing dyes described by Scott A. Hilderbrand and Stephen J. Lippard in “Cobalt Chemistry with Mixed Aminotroponiminate Salicylaldiminate Ligands: Synthesis, Characterization, and Nitric Oxide Reactivity” Inorganic Chemistry, (2004) 43(15), 4674-4682.

3) Optional Use of Nitrogen Dioxide (NO₂) Reactive Dyes.

NO reacts with molecular oxygen through the reaction 2NO+O₂→2NO₂. In the absence of a catalyst, the rate of this NO-consuming reaction increases with the square of the NO concentration. Therefore, at very high NO-concentrations NO is rapidly oxidized to NO₂. When this is the case, it is advantageous to detect NO₂ or to detect both NO and NO₂. Like NO, NO₂ can also be detected by a spectral or luminescence change. For example, T. Tanaka, et al. Sensors and Actuators, B: Chemical (1998), B47(1-3), 65-69 “Coloration reactions between NO₂ and organic compounds in porous glass for cumulative gas sensor” describe NO₂ requiring diazocoupling coloration reactions in the aqueous phase, which allow detection of NO₂ through absorbance changes. Similar reactions, but of water-insoluble, non-toxic aromatic amines such asp-phenylenediamine, 2,5-diaminotoluene, and couplers such as resorcinol, chlororesorcinol, methyl resorcinol, naphthols, m-aminophenol, m-phenylenediamine would allow NO₂ detection with lesser risk of leaching.

4) Forms of Reactive Materials

The reactive material is mounted on, attached to, coupled with, joined with or incorporated within the devices of the present invention. For example, when at least a portion of the device is comprised of silicone rubber, the reactive material 14 may be dissolved in and/or absorbed by the silicone rubber. In such instances the reactive material may be in solution form and taken up by soaking silicone rubber in the solution. Alternatively, the reactive material may be coated on a surface of the device, or adhered to an adhesive on a surface of the device.

Or, the reactive material may be mounted on or incorporated within a structure such as, for example, a tape, pad, mesh, or plate which is attached to the device, a structure such as a thread, strand, string, suture or filament which is wrapped around a portion of the device, or an inner, first sleeve into which at least a portion of the device may be inserted. In the example of a sleeve, the sleeve may be comprised of a polymer or a cellulosic material, which dissolves and/or adsorbs the reactive material. The reactive material containing inner first sleeve can be comprised of, for example, paper or a polymer such as polyvinyl acetate, partially hydrolyzed polyvinyl acetate, or polyvinyl alcohol.

Devices Having Exemplary Support Structures

1) Probes

It may be appreciated that the term probe may be used as a general term to describe all devices of the present invention. In addition, probe may be used to describe a specific type of device, such as a slender instrument used to explore a body cavity (American Heritage Dictionary, Second College Edition, Houghton Mifflin Company, 1995).

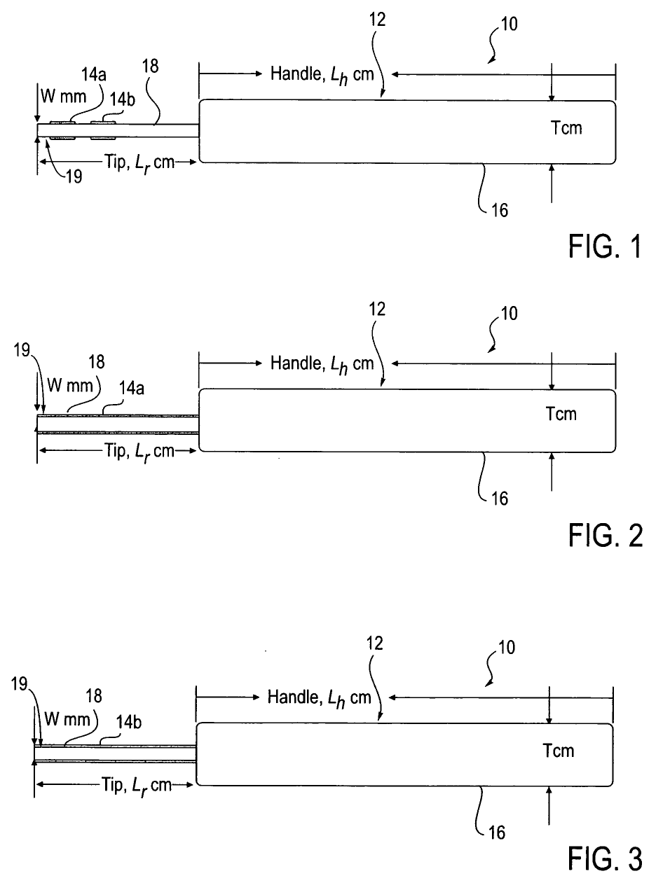

FIG. 1 illustrates an embodiment of a device 10 comprising a probe 12 and at least one reactive material 14. The probe 12 comprises a handle 16, configured for holding by one or more hands, and a tip 18, configured for insertion in or through an orifice of a body. The handle 16 is of a convenient length, Lₕ, for manipulation of the probe 12, typically between about 7 cm and about 20 cm. The diameter T of the handle is typically between about 1 cm and about 5 cm, preferably between about 1.5 cm and about 4 cm. The tip 18 is of suitable length, Lₜ, for insertion in or through an orifice for accessing a relevant gas-containing volume element of the body. The length Lₜ may vary depending on the intended usage of the probe 12. Typically the tip 18 is about 5-15 cm long for accessing the rectum or vagina. In some embodiments, the tip 18 is about 3-7 cm long for accessing the mouth. The diameter W of the tip 18, is typically smaller than that of the handle 16. In some embodiments, the diameter W is between about 2 mm and about 1 cm, preferably between about 3 mm and about 6 mm.

In the embodiment of FIG. 1, the at least one reactive material 14 is disposed on the tip 18 of the device 10. In particular, two reactive materials 14 are present, a color-changing material 14a and a fluorescence-changing material 14b. The materials 14a, 14b are illustrated adjacent to each other along the tip 18. However, it may be appreciated that the materials 14a, 14b may be disposed along the tip 18 in any configuration or pattern, such as in rings, strips, or blocks, to name a few, including overlapping and/or non-overlapping portions. Likewise, any number of materials 14 may be present and may be in repeating, such as alternating, or non-repeating patterns. In this embodiment, the materials 14a, 14b are shown near a distal end 19 of the tip 18, however the materials may extend to the handle 16. For example, FIG. 2 illustrates a probe 12 wherein the color-changing material 14a extends from the distal end 19 to the handle 16. And, FIG. 3 illustrates a probe 12 wherein the luminescence-changing material 14b extends from the distal end 19 to the handle 16.

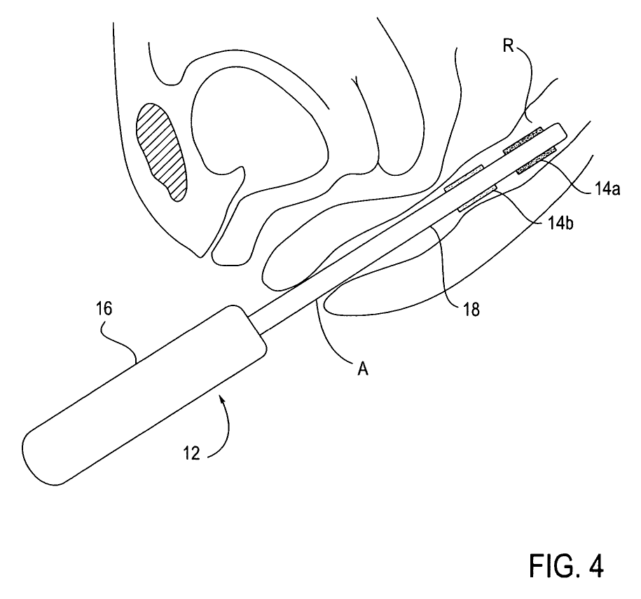

The probes 12 of FIGS. 1-3 have a form similar to a rectal, oral or vagina thermometer. Thus, the tip 18 is inserted into an orifice while the handle 16 remains outside of the body. Typically, the probed orifice of the body leads to a fluid-containing, preferably a gas-containing, volume element within which NO concentration is desired to be measured. For example, when probing NO in the luminal gas of the bowel, particularly the luminal gas in the colon or rectum, the orifice in which the probe is inserted is the anus. FIG. 4 illustrates a probe 12 as in FIG. 1 inserted in the anus A. As shown, the tip 18 extends into the rectum R so that the color-changing material 14a and a fluorescence-changing material 14b are positioned in the rectum R. The handle 16 remains outside of the body. It may be appreciated that the materials 14a, 14b may be positioned further into the bowel with the use of a longer tip 18, particularly a long, flexible tip 18.

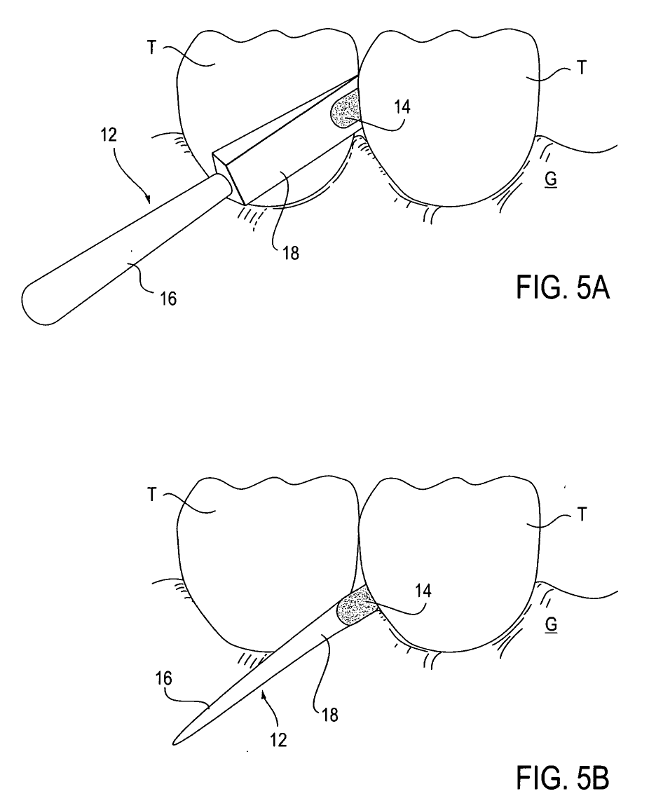

When probing NO in the upper respiratory tract, the mouth, the esophagus or the stomach, the orifice in which the probe is inserted is the mouth. When probing the mouth itself, the tip 18 may be placed at any location near a suspected diseased zone, including next to a tooth and between two teeth. When positioning between two teeth, the tip 18 is suitably sized and shaped for interdental insertion. For example, FIG. 5A illustrates an embodiment of a probe 12 inserted between two teeth T. Here, the tip 18 has a wedge-shape which narrows toward its distal end. The reactive material 14 is disposed along the tip 18 so that the material 14 is positionable between the teeth T and/or near the gums G. Likewise, FIG. 5B illustrates another embodiment of a probe 12 inserted between two teeth T. Here, the probe 12 resembles a toothpick. The tip 18 has a tapered cylindrical shape which narrows at each of its ends. The handle 16 is simply one end of the tip 18 portion. The reactive material 14 is disposed along the tip 18 so that the material 14 is positionable between the teeth T and/or near the gums G. Preferably, the probe 12 is comprised of a polymer or of wood in, or on, which NO-reactive material is immobilized.

The NO-reactive material is exposed at the site to be NO-tested for a pre-defined period of time, is withdrawn, is optionally rinsed and the spectral change is visually read, optionally using a calibration strip. Alternatively, the spectral change is instrumentally read. The probing part is inserted or applied for a predefined time period, is withdrawn and the spectral change is visually read, or is instrument read. The inserted probe 12 is kept in place and exposed for a period between about 1 sec and 1 hour, preferably between 10 sec and 20 min, and most preferably between about 30 sec and about 5 min. Alternatively, the spectral change is read during the exposure and/or after it in-situ, while at least part of the probe is in the body, the required optical components and electronic components, such as optical waveguides, or electrical connectors, or transmitters being incorporated in or attached to the probe.

It may be appreciated that other types of support structures may be used for dental or periodontal NO-testing. Examples include sheets and cords having reactive materials. FIG. 5C illustrates a cord 13 inserted between two teeth T. Here, the cord 13 resembles dental floss. The reactive material 14 is disposed along the cord 13 so that the material 14 is positionable between the teeth T and/or near the gums G. Thus, the cord 13 of FIG. 5C resembles the probe 12 of FIG. 5B and differs mainly by the material it is comprised of. Therefore, cords may be considered probes for the purposes of this application.

2) Tethered Bodies

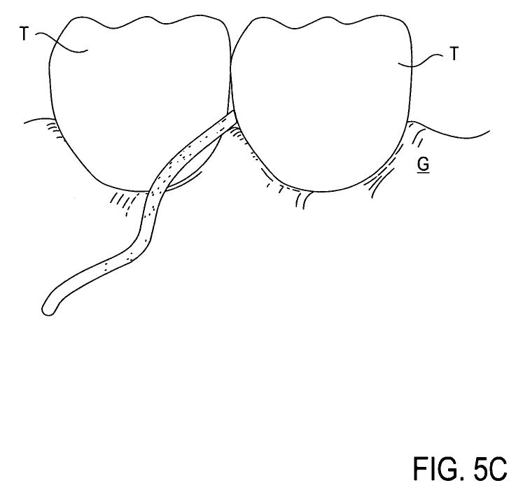

FIG. 6A illustrates an embodiment of a device 10 comprising a tethered body 30 and at least one reactive material 14. Here, two reactive materials 14 are present, a color-changing material 14a and a fluorescence-changing material 14b. The materials 14a, 14b are illustrated adjacent to each other along the tethered body 30. However, it may be appreciated that the materials 14a, 14b may be disposed along the tethered body 30 in any configuration or pattern, such as in rings, strips, or blocks, to name a few, including overlapping and/or non-overlapping portions. Likewise, any number of materials 14 may be present and may be in repeating, such as alternating, or non-repeating patterns. Further, a retrieving element 32 is attached to the tethered body 30. Such a retrieving element 32 is suitably long and strong enough to allow its retrieval. Further, the tethered body 30 may optionally be covered with an NO-permeable sleeve to reduce leaching of the NO-reactive material.

In some embodiments, the tethered body 30 of the present invention may be referred to as a plug, a feminine tampon or a suppository. In these embodiments, the tethered body 30, when suppository-like, typically has a diameter, h, between about 3 mm and about 2 cm, preferably between about 0.5 cm and about 1 cm. Its typical length, L, is between about 1 cm and about 4 cm, preferably between about 2 cm and about 3.5 cm. FIG. 6B illustrates such a tethered body 30 positioned within a vagina V in a manner similar to a tampon and having the typical dimensions of a tampon. In some of these embodiments, reactive material is incorporated into an outer portion of the tethered body 30 comprised of plastic. In other embodiments, the reactive material is included in a non-woven cover blend of a tampon described in K. Lochte et al. U.S. Pat. No. 6,758,839, wherein the reactive material containing part is optionally part of the tampon positionable near the cervix C. In any case, the capsule 30 is typically inserted into the vagina V for a period of about 20 min to about 8 hours and is visually or instrumentally checked for spectral change.

In other embodiments, the tethered bodies 30 of the present invention are swallowable wherein the a retrieving element 32 is suitably long and strong enough to allow retrieval of the tethered body 30 from the esophagus or stomach. In these embodiments, the tethered bodies 30 may be referred to as capsules or gastric capsules. In these embodiments, the tethered body 30 typically has a diameter, h, between about 1 mm and about 1 cm, preferably between about 2 mm and about 6 mm. Its typical length, L, is between about 4 mm and about 2 cm, preferably between about 6 mm cm and about 1.2 cm.

FIG. 6C illustrates such a swallowable tethered body 30 wherein the body 30 is positioned in the stomach ST of a patient P. The reactive material may be incorporated into an outer portion of the body 30 comprised of plastic. The body 30 is swallowed while its retrieving element 32 remains outside the body, such as held by the hand of the patient or physician, or attached to an external entity, such as an external part of the patient, such as the wrist or the waist. Optionally, passage of the body 30 past the stomach ST is prevented by providing a retrieving element 32 long enough for residence in the stomach ST, but not beyond it. The body 30 is retrieved, typically after a period between about 3 min and about 1 hour, preferably after a period between about 5 min and about 30 min and is visually or instrumentally checked for spectral change.

3) Beads

Beads are configured for passage through the body, rather than by retrieval with the use of a retrieving element. For example, a bead may be swallowed by a patient and recovered from the patient's feces. Therefore, beads are typically round, spherical, oval, ellipsoid or oblong without sharp edges or corners. Each bead is typically larger than about 0.01 cm in its smallest dimension, and is typically smaller than about 2 cm in its largest dimension. Each bead is preferably larger than about 0.1 cm in its smallest dimension and smaller than about 0.5 cm in its largest dimension.

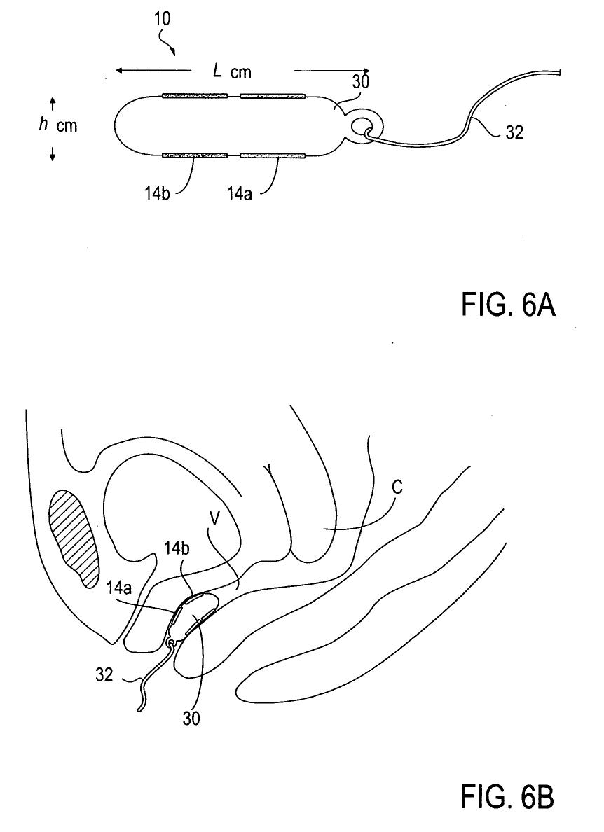

FIG. 7 provides a cross-sectional view of a bead 40 having a core 42 of a magnetic metal and/or a magnetic metal oxide. The core 42 is coated with a reactive material 14. Typically the reactive material 14 is incorporated into a plastic, a ceramic, or a composite which surrounds the core 42. In preferred embodiments, reactive dye is incorporated into a plastic, and is most preferably an elastomer, such as a rubbery poly (dimethyl siloxane) also commonly referred to a silicone rubber. Prior to its dying with the reactive material the magnetic core-coating plastic is preferably colorless. It can be optically transparent, or translucent, or white, white meaning that it comprises a colorless pigment, having an index of refraction higher than that of the plastic, so as to scatter light. An exemplary non-toxic pigment is titanium dioxide of a preferred average particle size between about 50 nm and about 500 nm.

Such beads may be used to analyze the entire digestive tract. When in a mixture and/or in feces, the magnetic bead can be separated and/or collected with a magnet or electromagnet.

Alternatively, the spectral change of the beads may be monitored in-situ during the exposure and/or after it, while the bead is in the body or in the feces, the required optical and electronic components, including at least one light source, at least one detector, and an RF transmitter being in or attached to the bead. In this case the magnetic core may be omitted.

4) Sheets

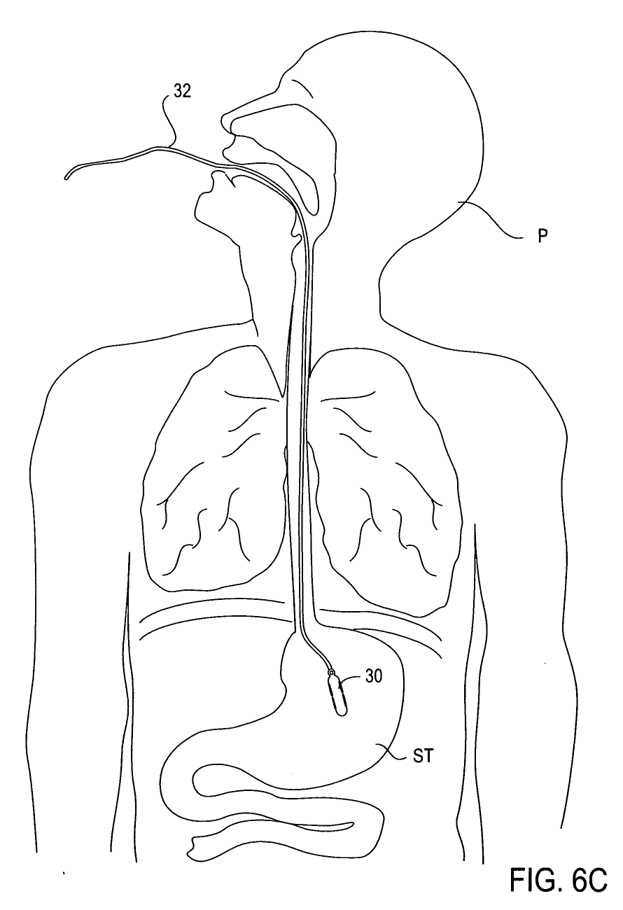

In preferred embodiments, sheets are configured for measuring NO concentration on or emanating from a surface of a tissue, such as skin. FIG. 8 illustrates an embodiment of a sheet 50 with a reactive material 14 therein. The sheet 50 is shown applied to the skin S of an arm of a patient. Typically, the sheet 50 comprises paper, cloth, plastic or other suitable material which is able to be applied to the tissue surface. The sheet 50 may also include adhesive to adhere the sheet to the tissue surface. Such sheets 50 may be used to test for disease of the skin. The typical thickness of a sheet 50 that is adhered to a skin surface is between about 0.1 mm and about 3 mm, preferably between about 0.4 mm and about 1.2 mm.

Prior to its dying with an NO-reactive material, the sheet 50 is preferably colorless. In preferred embodiments, sheet 50 is comprised of plastic and is optically transparent, or translucent, or white, white meaning that it comprises a colorless pigment, having an index of refraction higher than that of the plastic, so as to scatter light. An exemplary non-toxic pigment is titanium dioxide of a preferred average particle size between about 50 nm and about 500 nm.

The material of the sheet 50 preferably at least partially adheres to the skin, most preferably also to the wet skin, yet is removed with minimal pain or no pain. Exemplary materials are those such as used in wound dressings, such as plastics and structures adhering to wet skin described by D. H. Lucast et al. U.S. Pat. No. 6,198,016 and U.S. Pat. No. 6,518,343, skin adhesive pressure sensitive blends comprising hydrophilic and hydrophobic components, including copolymers of (meth)acrylate esters, described by P. D. Hyde et al., U.S. Pat. No. 6,497,949, and low-trauma adhesive wound dressings that are easy to remove, their removal causing little or no pain, described by E. G. Joseph et al. U.S. Pat. No. 6,171,985 and U.S. Pat. No. 6,368,687. Exemplary materials available in pharmacies include Johnson and Johnson Non-Irritating Paper Tape Dermatologically Tested for Sensitive Skin, available from Johnson and Johnson Consumer Products Company, Skilman, N.J., and Nexcare® Gentle Paper First Aid Tape, available from 3M, St. Paul, Minn.

It may be appreciated that sheets 50 may be applied to any tissue surface, such as luminal surfaces of the body. Further, sheets 50 may be positioned between teeth, such as a probe 12, as described above.

External Coverings

The devices 10 of the present invention may be constructed of any suitable material or combination of materials, such as plastic, metal, ceramic or a composite. Because of the lower cost of plastics, a plastic, or a material comprising a plastic, is preferred. The reactive material 14 is mounted on, attached to, coupled with, joined with or incorporated within the device by any appropriate means, such as described above.

In addition, the device 10 or portions of the device may optionally be covered by a covering such as a coating, a thin polymer film, a sheet, overlay or a sheath, to name a few. The covering reduces or prevents leaching of the reactive material or its reaction product into the biological environment and/or the covering reduces the possibility of adverse reaction, such as allergic reaction, to one or more components of the device 10. For example, the toxicological or biological properties of all reactive material and/or products of their reactions may not be as thoroughly investigated as desired in order to allow their contact with and/or leaching by fluids and/or cells of tissues of the body. Further, some reactive materials can be water soluble and therefore soluble in a body fluid. To eliminate or reduce the likelihood of potential harm, the device 10 or, in particular, portions of the device 10 including reactive material 14, may be covered with a covering.

The covering is highly permeable to the reactive chemical compound, such as NO, but is impermeable or is much less permeable to the reactive material and/or its product. It is desirable that the ratio of the permeation rates of NO and/or the reactive material and/or of NO and the reaction product be greater than 10, preferably greater than 100, and most preferably greater than 1000. Usually the covering dissolves less than 1/100^{th} ooth of a percent by weight of the reactive material when exposed to its about 0.1 M solution in a solvent in which the covering does not measurably swell, but dissolves at least 1/10^{th} of a percent by weight of NO and/or NO₂ at 1 atm pressure of either of these gases.

Typically, the covering is thin enough to assure that when used as a membrane to separate two compartments of equal volume, both at 37° C., the difference in the partial pressure of NO between the two compartments is less than about 10 percent after about 1 min. An exemplary covering comprises a coating of an elastomeric silicone or silicone rubber, such as an elastomeric poly(dimethylsiloxane) comprising film. The characteristic diffusion coefficient, D_{c}, of NO in elastomeric silicone is about 4×10⁻⁵ cm² s⁻¹. The characteristic diffusion length, L_{c}, for τ_{c=}1 min, is about 0.5 mm. Thus, a particularly useful coating thickness range is between about 50 and 500 micrometers, and a thickness between about 100 and about 200 micrometers is preferred. When the device 10 includes a light guide, the coating may form at least a portion of the light guide. In such instances, the device 10 is comprised of a plastic having an index of refraction higher than that of the applied coating.

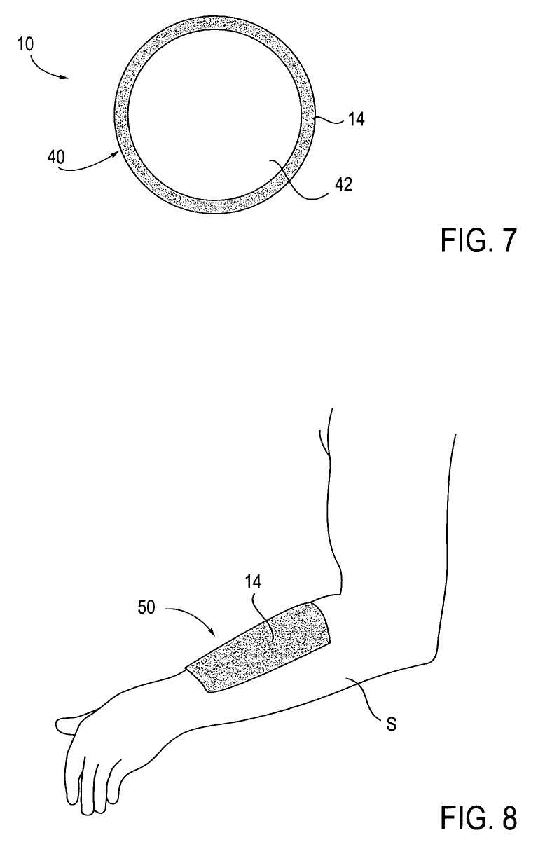

In some embodiments, the covering comprises a thin sleeve or sheet, such as comprised of a silicone rubber or other elastomeric material, in order to avoid or reduce the leaching of the reactive material 14 into the contacting fluid and/or to reduce any possible adverse, for example allergic, reaction to a material of the device, as described above. FIG. 9 illustrates an embodiment of such a covering having the form of an external sleeve 60. Here, a probe 12 is shown wherein the tip 18 is inserted into the sleeve 60. The reactive material 14 is disposed on or within the tip 18 which is covered by the sleeve 60. The reactive chemical compound, such as NO, is able to permeate the sleeve 60, as indicated by arrows 61, so as to react with the reactive material 14, however the reactive chemical compound is not able to rapidly pass through the sleeve 60, as indicated by return arrows 62. The sleeve 60 can be comprised of, for example, an elastomer, such as a silicone rubber, or of a low-density, non-crystalline polymer. Typically, the thickness of the sleeve 60 is less than about 2 mm and is 10 preferably less than about 1 mm. The sleeve 60 is typically removable, allowing optional re-use of the device 10. The devices 10 can be self inserted and retrieved by the patient, or they can be inserted and retrieved by a health professional. Devices 10 which include irreversibly reacting materials that are used only once and are discarded after use. Devices 10 having reversibly reacting materials can be used more than once. Therefore, the inserted parts of the device may be sheathed with a removable elastomeric sleeve, such as an elastomeric silicone sheet thinner than about 0.5 mm, to avoid the need of sterilization between uses. Instead of sterilization, the sleeve is replaced.

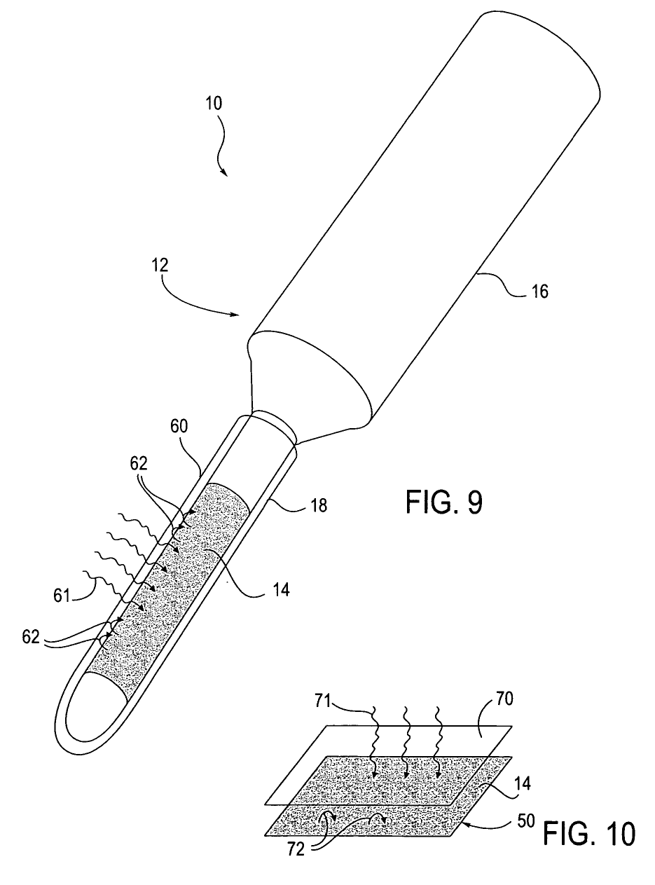

FIG. 10 illustrates an embodiment of such a covering having the form of an external sheet 70. Here, a sheet 50 having the reactive material 14 disposed therein or thereon. The sheet 50 is covered by the external sheet 70, as shown. The reactive chemical compound, such as NO, is able to permeate the sheet 70, as indicated by arrows 71, so as to react with the reactive material 14, however the reactive chemical compound is not able to pass through the sheet 70, as indicated by return arrows 72. The sheet 70 can be comprised of, for example, an elastomer, such as a silicone rubber, or of a low-density, non-crystalline polymer. The sheet 70 may optionally be removable.

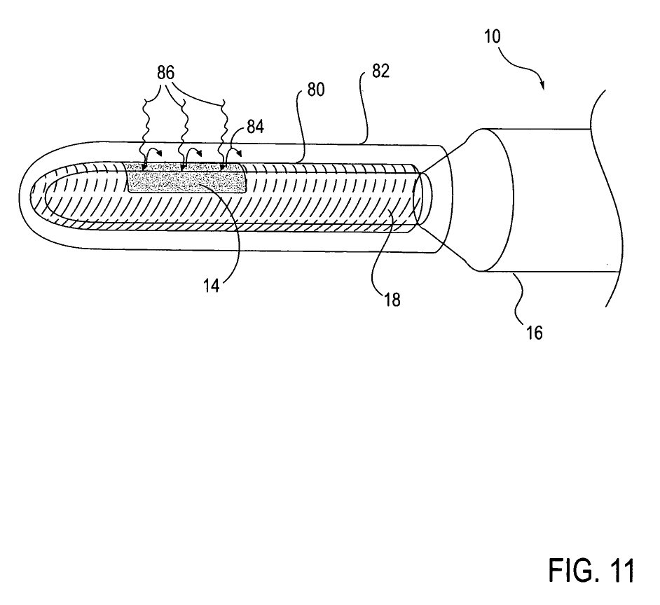

Referring to FIG. 11, it may be appreciated that the when the reactive material 14 is disposed on an inner, first sleeve 80 into which at least a portion of the device 10 is inserted (such as tip 18), an additional outer, second sleeve 82 may be present. As mentioned above, the inner, first sleeve 80 may be comprised of a polymer or a cellulosic material, which dissolves and/or adsorbs the reactive material 14. An additional outer, second sleeve 82 may be placed over the inner, first sleeve 80 in order to avoid or reduce the leaching of the reactive material 14 into the contacting fluid and/or to reduce any possible adverse, for example allergic, reaction to a material of the device 10. Prevention of reactive material 14 passing through the outer, second sleeve 82 is indicated by return arrows 84. However, the reactive chemical compound, such as NO, may easily pass through the outer second sleeve 82, as indicated by arrows 86. It may be appreciated that the sleeves 80, 82 are shown loosely fitting for illustration purposes only and may be fit at any level of snugness against the tip 18 and/or each other.

In some embodiments, the covering comprises a conformal NO-permeable coating to reduce or eliminate leaching of the reactive material and to prevent adverse reaction, such as allergic reaction. Conformal coatings are protective materials applied in thin layers, typically about 0.05-1 mm, and are commonly used on printed circuits and on other electronic substrates. Their materials are typically acrylics, urethanes or silicones. Of these, non-toxic elastomeric silicones are preferred. These silicones can range from tough, abrasion resistant materials known as elastoplastics to soft, stress relieving, rubbery elastomerics. Conformal silicone coatings are typically applied by dipping, spraying or flow coating and cure, in normally humid, about 40-90% relative humidity, ambient air, at room temperature. Their curing is significantly accelerated by heat, even mild heat. Conformal clear or translucent elastomeric silicone coatings, such as those available from Dow Corning of Midland, Mich., are most preferred.

Use of the Devices

The devices 10 can be used, for example, to screen patients, meaning to determine whether they should be further tested by more expensive procedures, methods or instruments, such as endoscopy, colonoscopy, magnetic resonance imaging. Devices 10 can also be used to determine whether they should be treated, for example for inflammation. Devices 10 can be further used to determine whether they respond to treatment, a favorable response being indicated by lesser or slower spectral or luminescence change of the reactive material 14.

1) NO-Exposure, Diffusion, Concentration, Reaction Time and Local Change of the Spectrum.

The half life of NO can be of many minutes, even many hours or days, long enough for the NO to diffuse to the reactive material 14 or to penetrate a sleeve or coating, preferably amorphous or elastomeric plastic, to react with the reactive material, causing change in its spectrum. Usually, the higher the NO concentration, the more rapid is the change in the spectrum. The NO-concentration is estimated by observing the spectral change and/or the rate of the spectral change. An increase in NO concentration is indicative of need for additional testing, and/or of active disease. Following treatment, a decrease in NO-concentration usually indicates that the treatment was effective. The NO-concentration typically increases with the severity of inflammatory disease, typically decreases upon its effective treatment. Thus, the spectral change and/or rate of spectral change are also of prognostic value.

NO diffuses most rapidly in the gas phase, but it also diffuses in aqueous solutions and in lipophilic solvents. It permeates through membranes of cells, the skin, and plastics. It permeates readily through elastomeric polymers, such as silicone and other rubbers, and through amorphous, particularly non-relaxed, thermoplastic polymers. Because spectral change is produced by the reaction of NO-reactive material in or on the plastic, the exposure is NO-flux and time dependent.

The NO-concentration, the NO-flux, the distance between a NO-reactive material-containing device site from the NO-generating diseased tissue and the exposure time are related by the two Fick equations, found in chemical engineering textbooks and textbooks on diffusion through solids, such as polymers. According to Fick's first law, the net diffusion rate of NO, for example across a membrane or a space element, is proportional to the difference in partial pressures, proportional to the area of the gaseous, liquid or solid material through which NO diffuses, and is inversely proportional to the thickness of the material in which the NO diffuses. Its relative rate of diffusion is proportional to its concentration, which scales about linearly with its partial pressure. From Fick's first law the flux, J, of NO at concentration, C, across a plane of unit area, is proportional to the concentration gradient ΔC/Δx that plane and is expressed by:

$$J = - D \frac{\partial C}{\partial x}$$

where D is the diffusion coefficient. According to Fick's second law the rate of change of concentration in a volume element of a membrane, within the diffusional field, is proportional to the rate of change of concentration gradient at that point in the field, as given by:

$$∇ ( D ∇ C ) = \frac{\partial C}{\partial t}$$

For a constant D, the rate of change in concentration with time is proportional to the rate at which the concentration gradient changes with distance in a given direction, i.e.:

$$\frac{\partial C}{\partial t} = D \frac{{\partial }^{2} C}{\partial {x}^{2}}$$

It can be shown that the steady-state concentration of NO ([C]) as a function of distance (Δx) away from its source, which is, for example, the malignant tumor or the inflamed tissue in which its concentration is [C]₀, when the half-life of NO in the phase in which it is least permeable is t_{1/2}, is given by

$$[ C ] = {[ C ]}_{0} {{ⅇ}^{- Δ x} ( \frac{ln 2}{{D}_{c} {t}_{1 / 2}} )}^{1 / 2} ,$$

where D_{c} is the effective diffusion constant in the phase or phases through which the NO diffuses before it reacts with the NO-reactive material.

Thus, when the concentration of NO is higher, its flux and reaction rate with the NO-reactive material, are faster. As a result, the exposure in a predefined time period, and the spectral change, increase. Also, when a diseased site is closer to a particular NO-reactive material-containing zone of the device, the exposure in a predefined time period, and the spectral change, also increase. Furthermore, when the NO-concentration is higher at the device, a lesser exposure period is required to achieve a pre-defined spectral change, such as the bleaching of an NO-reactive material like PTIO, or an increase in the fluorescence intensity of a NO-reactive material like DAA, II, IV or VI. Therefore, when the NO-source is closer to the device, and/or when the NO-concentration at the source is higher, the spectral change is faster and the change in a given period of time is greater.

2) Determination of the Direction of a Diseased Zone with Respect to the Device

The spectral change is determined by the exposure. Unless most of the NO-reactive material molecules have already reacted, the greater the number of NO-molecules reacting with the NO-reactive material, the greater the spectral change. This provides a means for determining the direction of the diseased zone with respect to a set of separated NO-reactive material-containing probe zones. The closer a zone of the probe is to the diseased tissue, the greater the exposure, and, consequently, the spectral change. For example, if the NO-reactive material is PTIO, than the blue color of the dyed zone most bleached by the NO is that closest to the diseased tissue. If the NO-reactive material is compound I, compound III, compound V, 2,3-diaminonaphthalene, or 1,2-diamino-anthraquinine (DAA) then the fluorescence is most intense in the part of the device that is closest to the diseased tissue.

3) Spectrum Reading

The device 10 is exposed to NO or other measured gas, and its exposure is read in situ, or the device 10 is withdrawn for reading. The change in the spectrum is visually or instrumentally read. In the simplest embodiment, the spectral change is observed after exposure by the naked eye. Optionally, a calibration or reference strip is used to quantify the seen spectral change. This method is preferred for the self-monitoring patient and/or for monitoring at home.

Alternatively, the spectral change and/or the rate of change is read with an instrument, or with a system formed of two or more instruments. The instrument, and in the system at least one of the instruments, comprises a light source and a detector, and optionally comprises one or more filters, and/or one or more lightguides, and also optionally uses phase-sensitive detection. Optionally, the components are packaged with at least one battery and a display in the handle of the device, to form a handheld, autonomous, NO-monitoring system. Alternatively, the components are integrated in the handle 16 with at least one battery and an RF transmitter, to form an NO-sensor/transmitter, the RF receiver/recorder and/or display being located usually within about 50 meters, preferably within 20 meters and most preferably within 10 meters of the patient.

4) Threshold NO-Concentration for Referral for Further Testing

In screening, for the purpose of lessening the physical discomfort and/or the monetary burden of the cost of upper GI endoscopy and/or colonoscopy, and/or the monetary burden of the cost of fecal DNA assay, and/or for including in the screened population also the majority of the people older than 50, who are in need of screening but are not screened, sensitivity is more important than selectivity.

To increase sensitivity, meaning to reduce the likelihood of missing the detection of an existing disease, lesser selectivity, meaning greater signaling of disease in healthy people, is accepted. For this purpose, the threshold NO-concentration at which further testing is recommended to a patient is preferably set between about 0.9 times the upper limit of the NO concentration in healthy individuals and about 2 times this upper limit. For example, in screening for disease of the colon or the rectum, the preferred threshold above which patients are referred for colonoscopy and/or DNA testing is between about 180 ppb and about 360 ppb.

Diseases Diagnosed

The diseases that may be diagnosed using the devices and methods of the present invention are preferably those where the diseased tissue is in contact with a cavity, the cavity containing a gas, exemplified by the following:

1) Neoplasms and/or Inflammation of the Gastrointestinal System

Particularly those exemplified by, or associated with, Barrett's esophagus, gastric polyposis, gastric intestinal metaplasia, gastric carcinoma, gastric adenocarcinoma, gastric mucosa-associated lymphoid tissue lymphoma, Helicobacter pylori infection, gastritis, ulcerative gastritis, Peutz-Jeghers syndrome, juvenile polyposis, familial polyposis, chronic ulcerative colitis, Crohn's disease of small and/or large intestine, non specific inflammatory bowel disease, irritable bowel syndrome (negative information), familial history of colonic neoplasia and/or neoplastic polyps of the colon, food intolerance, exemplified by lactose intolerance.

2) Neoplasms of the Mouth

Particularly those exemplified by, or associated with, leucoplakia of the mouth, and/or white lesions of the mouth.

3) Neoplasms and/or Inflammation of the Female Reproductive System

Particularly those exemplified by, or associated with, chronic cervicitis, dysplasia of the cervix, and/or endometrial neoplasia.

4) Neoplasms of the Skin

Particularly those exemplified by or associated with skin neoplasia, such as melanoma.

5) Neoplasms and/or Inflammation of the Respiratory System

Particularly those exemplified by, or associated with, inflammatory and neoplastic diseases of the lungs; nodules of the larynx; inflammatory conditions of the nasopharynx and larynx and/or smoking.

Exemplary Clinical Procedures

1) Use of a Thermometer-Like Rectal Probe

The tip of the probe is inserted in the rectum to about 3-15 cm depth and retrieved after about 2-20 min for visual or instrumental reading, or if integrated with the optical components and an RF transmitter, or integrated with the optical components and connected with a fiber optic or electrical cable to a monitoring system, the spectral change is tracked while the tip is inserted.

2) Use of the Gastric Capsule

The test for inflammation and/or neoplasia, for example carcinoma, of the stomach may be performed with food in the stomach or, optionally in the morning before breakfast when the stomach is contains little or no food. The patient is asked to swallow the capsule, optionally by drinking a small cup of water, about 50 mL to about 100 mL, while the retrieving element is held in his or her hand, or, particularly in the case of a child, is tied to the waist or to another non-moving part of the body. The capsule is retrieved, typically after a period between about 3 min and about 1 hour, preferably after a period between about 5 min and about 30 min and is visually or instrumentally checked for spectral change.

3) Use of a Colonoscopy-Associated NO-Probe

The colon is empty for the test, as it is in colonoscopy. The patient is usually on a clear liquid diet, for 1 to 2 days beforehand, and is given more laxatives the night before the procedure. As in colonoscopy, the patient lies on the side, preferably the left side, on the examining table. The probe is about 2 m long and flexible, and comprises the NO-reactive material in its 10-40 cm long front part. The probe is inserted into the colon through the biopsy channel of the colonoscope. To test for disease, the NO-reactive material containing tip is moved from the rectum, through the colon, to the lower end of the small intestine. The probe retrieved, rinsed and visually or instrumentally checked for spectral change. If none is observed, the test suggests absence of severe disease. If a change is observed, the test is repeated, but now the probe is inserted as rapidly as is practical, to the zone propped, held there preferably for about 3-5 minutes and is rapidly retrieved, rinsed and is visually or instrumentally read. Alternatively, if anything abnormal is seen in the colon in the process of colonoscopy, like a polyp, or inflamed tissue, or neoplasia, the probe is moved to the aberrant site, and is preferably held there for about 1-5 minutes, to test for disease. It is then retrieved, rinsed and is visually or instrumentally read for spectral change.

4) Use of an ERCP-Associated NO-Probe

For NO-probing with endoscopic retrograde cholangiopancreatography (ERCP), to diagnose inflammation or neoplasia in the liver, gallbladder, bile ducts and pancreas, the stomach and duodenum are empty. The patient is asked not to eat or drink anything after midnight the night before the procedure, or for 6 to 8 hours beforehand, depending on the time of the procedure. For the procedure, the patient lies on the side, preferably the left side, on an examining table, swallows the endoscope, and the physician guides the scope through the esophagus, stomach, and duodenum until it reaches the spot where the ducts of the biliary tree and pancreas open into the duodenum. At this time, the patient is turned to lie flat on his abdomen. The endoscopist passes the NO-reactive material containing probe, of dimensions and characteristics similar to the one used for colonoscopy, through the scope, holding the NO-reactive material-containing tip for 1-5 min at the suspect site, then retrieving it, rinsing it and reading it visually or instrumentally.

5) Use of a Flexible Sigmoidoscopy-Associated NO-Probe

For flexible sigmoidoscopy with NO-probing the colon and rectum are preferably empty, the patient is asked to use 2 enemas containing phosphosoda 2 hours prior to the procedure. For the procedure, the patient lies on the left side. The physician inserts the sigmoidoscope and examines the rectum and colon, then if anything unusual is observed, like a polyp or inflamed tissue, the physician inserts in the scope the NO-probe, of dimensions and characteristics similar to the one used for colonoscopy, guiding its NO-reactive material-containing end to the suspect side, keeping it there preferably for about 1-15 min, retrieving it, rinsing it and visually or instrumentally observing the spectral change.

6) Use of an EGD-Associated NO-Probe

For upper endoscopy, also termed esophagogastroduodenoscopy (EGD), the stomach and duodenum are usually empty enabling the physician to look inside the esophagus, stomach, and duodenum. The patient is usually told not to eat or drink anything for at least 6 hours beforehand. The patient swallows the endoscope which transmits an image of the inside of the esophagus, stomach, or duodenum, allowing the physician to examine the lining of these organs. If a suspected abnormality is seen, the physician tests it disease by inserting the NO probe, of dimensions and characteristics similar to the one used for colonoscopy, keeping its NO-reactive material-containing part at the suspect site for about 1-15 min, retrieving it, rinsing it an visually or instrumentally observing the spectral change associated with the elevated NO concentration.

7) Use of a Retrievable Rectally Inserted Suppository

The NO-monitoring suppository is optionally patient inserted and kept inside for about 1 min—about 2 hours, then is retrieved for observation of color change or fluorescence change. The reading can be visual or instrumental. When deeply inserted, it is preferred that the suppository be inserted by a health professional.

8) Use of Magnetic Beads

The patient is instructed to swallow the NO-reactive material-containing magnetic beads and to recover part of these, using a plastic rod with a magnetic tip, from the feces. The recovered beads are rinsed by the patient and are brought to or are sent to a health professional for examination for spectral change.

9) Protocol for Testing Barrett's Esophagus

The exemplary NO-reactive material comprising device is about 2 mm diameter outer diameter, about 2 m long. It is optionally a silicone or polyester or nylon monofilament, stiff enough to be easily threaded in 2 m long tube. It is inserted in the esophagus, exposed for about 90 sec, withdrawn and the spectral change is observed visually or determined instrumentally. Positive for neoplasia shows spectral change immediately after exposure; actual reading of the change 5 min after end of exposure; color photography at conclusion of the endoscopy.

10) Other Test of the Digestive System

The NO testing is useful in discriminating between NO-values associated with certain diseases, their severity and diseases entering remission. For example, because the NO-levels are high but non-identical, it discriminates between Crohn's disease and ulcerative colitis, and they tell objectively whether the disease is going into remission. This is also the case for other inflammatory conditions of the colon, such as microscopic colitis. The NO test also discriminates between benign and malignant masses of the colon, between stomach diseases such as ulcers, and benign and a malignant tissue of the stomach, exemplified by intestinal metaplasia of the stomach, and/or gastric polyposis.

11) In Testing for Disease of the Mouth

Exemplified by, or associated with, leucoplakia of the mouth, an/or white lesions of the mouth the patient is told to hold the NO-reactive material-comprising tip of the thermometer-like probe in the mouth. The tip is retrieved after 1-20 min for visual or instrumental reading, or if integrated with the optical components and an RF transmitter, or integrated with the optical components and connected with a fiber optic or electrical cable to a monitoring system, the spectral change is tracked while the tip is inserted.

12) In Testing for Periodontal Disease

An NO-reactive material comprising device, such as a toothpick or dental floss, is inserted between the teeth and kept in place typically for a period between about 1 min and 1 hour, then retrieved for reading.

13) In Testing for Disease of the Skin

A NO-reactive material containing sheet of paper, cloth or plastic is placed over, and/or is adhered to the skin zone to be tested, kept in place for a period between about 2 min and about 20 hours, preferably between about 5 min and about 10 hours and either periodically examined, or examined once at the end of the test period, for spectral change at the site overlapping the suspect zone of the skin.

14) Testing for Disease of the Female Reproductive System

In using a thermometer-like vaginal probe, the tip is inserted in the vagina to about 4-15 cm depth and retrieved after about 2-20 min for visual or instrumental reading, or if integrated with the optical components and an RF transmitter, or integrated with the optical components and connected with a fiber optic or electrical cable to a monitoring system, the spectral change is tracked while the tip is inserted. Tests for diagnosis and/or treatment of diseases exemplified by, or are associated with, chronic cervicitis, dysplasia of the cervix, and/or endometrial neoplasia, pelvic inflammatory disease, carcinoma of the cervix, malignant and pre-malignant lesions of the vulva such as lichen sclerosus et atrophicus, craurosis vulvae, or uterine polyps and fibroids can be performed, for example, with the thermometer-like probe when the test period is shorter than about 20 min, and with the NO-detecting tampon when it is longer. In menstruating women, the NO-detection is preferably performed in the part of the non-menstrual part of the cycle, for example during the first week or second week after the menstrual period. The NO-reactive material containing tampon is inserted for a period of about 20 min to about 8 hours and is visually or instrumentally checked for spectral change.

For hysteroscopy, a flexible plastic probe containing the NO-reactive material is used. The NO-sensing probe-containing or an NO-sensing probe-modified modified hysteroscope is inserted into the uterus through the vagina and cervix. Typically the probe is about 1-10 mm in diameter, 10-50 cm long and its typically 1-10 cm end segment contains the NO-detecting NO-reactive material. In menstruating women, the NO-detection is preferably performed during the first week or after the menstrual period. It may be performed without anesthesia, or with local, regional or general anesthesia, optionally with the opening of the cervix dilated and without, or optionally with, a gas released through the hysteroscope to expand the uterus. The probe is inserted, with the NO-reactive material-containing region proximal to the tested zone, for a period preferably between about 1 min and about 20 min, then withdrawn, rinsed with water and visually or instrumentally tested for spectral change.

15) Bronchoscopy with NO-Testing for Disease of the Respiratory System

The bronchoscopy is performed by a medical professional, preferably by a pulmonologist, who tests for inflammatory and neoplastic diseases of the lungs; nodules of the larynx; inflammatory conditions of the nasopharynx and larynx and/or smoking. The patient typically fasts, preferably for 6 to 12 hours before the test. The bronchoscopy is performed usually via the nose. An anesthetic jelly is inserted into one nostril; when the nostril is numb, the flexible bronchoscope, its tube preferably less than about 1.25 cm in diameter, and between about 30 cm and 1 m long, is inserted. The NO-probe, similar to that used for colonoscopy, except only between about 30 cm and about 1 m long, is inserted through passed through a channel of the bronchoscope into the lungs to the probed zone, held at the site for between about 30 s min and about 10 min, the patient being instructed to hold his/her breath for the tests of less than about 90 s and to breathe out slowly for the longer tests. The probe is then withdrawn and is visually or instrumentally read. This test is preferably repeated once or more times.

16) Screening the Gastrointestinal System for Disease by Probing the Luminal Gas.

In the gas of the rectum or the colon of healthy people the NO-concentration is about 50-200 ppb. For sensitive diagnosis of elevation of the NO concentration up to about 5 ppm, monitoring of change in fluorescence, for example with compound I, III, V, or a vicinal diamine comprising NO-reactive material is preferred. For diagnosis of drastic elevation of the NO concentrations, to about 5 ppm-100 ppm, monitoring of change in the absorption or the reflection spectrum, for example with PTIO, is preferred.

Because NO diffuses rapidly in the gas phase, a major part of the gastrointestinal system, its entire length through which the gaseous lumen passes, can be probed for disease by a rectal probe, even though the probe is at a substantial distance from the probed tissue. Thus using the rectally inserted spectrum-changing NO-probe, the entire colon and the entire rectum is probed. The inserted part of the probe is at a depth of about 1-20 cm, preferably about 3-18 cm and most preferably about 5-15 cm. Inserted parts of different shapes and sizes can be used. The preferred inserted part shapes are elongated, have preferably no sharp edges or corners, and the ratio of their length to maximum width is greater than about 1, preferably greater than about 2 and most preferably greater than about 3. Their diameter at their maximum width is generally greater than about 2 mm and less than about 3 cm, preferably greater than about 3 mm and less than about 2 cm and is most preferably between about 4 mm and about 1.5 cm. The inserted parts are generally longer than about 5 mm and are shorter than about 15 cm; they are preferably longer than about 1 cm and shorter than about 15 cm; and most preferably are longer than about 2 cm and are shorter than about 10 cm. The part is generally shaped for painless insertion and removal. An exemplary inserted part is cylindrical with all edges rounded. The NO-reactive material can be in the inserted part, near the surface or on the surface of the inserted part, or it can be in a sleeve mounted on the core of the inserted part. The core of the inserted part, which defines the mechanical properties, is stiff enough for ease of insertion.

### EXAMPLES

### Example 1

### Probe Materials in Which Blue PTIO was Bleached by NO

2-phenyl-4,4,5,5-tetramethylimidazoline-1-oxyl 3-oxide (PTIO) was purchased fro Sigma-Aldrich Corp., St. Louis, Mo. FDA (a) White silicone rubber probe, 0.093 inches in diameter part # SC6020204 was purchased from Ipotec Inc., Exeter, N.H. The part of the probe to be dyed was pre-soaked for 10 min in tetrahydrofuran (THF). About 6 mg of the PTIO were dissolved in about 10 mL of THF. The pre-soaked probe was then immersed in the PTIO solution for 5 min and allowed to dry for 24 h. Upon soaking, the silicone rubber turned blue. Its blue NO-reactive material was not removed by wiping, even when the wiping tissue was wetted with THF. Nitric oxide was generated in a vial by mixing equal volumes of aqueous solutions of about 1 M FeSO₄ and about 0.5 M NaNO_{2.} An about 1″ long segment of the dyed part of the probe was exposed for about 20 s to the NO-containing gas. The exposed segment was bleached, loosing its blue color. (b) As in (a), except that a ⅛″ diameter OD translucent silicone dimethylsiloxane rubber mono-filamentary probe was used and the PTIO solution was applied with a dropper containing the THF solution touching the rubber in 5-6 passes. (c) As in (b), except that a 3/16″ OD white woven probe of polypropylene-polyester was used. (d) As in Example (b), except that a ⅛″ colorless woven probe of nylon-6, 6 and polyester was used. Only the polyester became blue. (e) A sheet of “Non-Irritating Paper Tape Dermatologically Tested for Sensitive Skin”, made by Johnson and Johnson Consumer Products Company, Skilman, N.J. was died blue with an about 0.05 weight % solution of PTIO in acetone then air dried. When the sheet was held over the mouth of a vial in which nitric oxide was generated by reacting dissolved FeSO₄ with also dissolved NaNO₂, the circular part of the sheet over the mouth of the vial turned yellow. The blue to yellow change was visible on both sides of the sheet.

### Example 2

The paper tape of Example 1 (e) was adhered to an inflamed cut in the skin of a volunteer for about 10 hours. The inflamed region of the cut was precisely mapped and was clearly visible as a colorless domain in the blue tape.

### Example 3

The paper tape of Example 1 (e) was adhered to the front end part of an endoscope probe and applied in colonoscopy. The paper turned colorless in the typically 1-3 minute long procedure in patients with inflammatory bowel disease revealed by parallel colonoscopy or endoscopy.

All publications, websites, patents and patent applications cited in this specification are herein incorporated by reference as if each individual publication or patent application were specifically and individually indicated to be incorporated by reference.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A device for evaluating a suspected diseased tissue of a patient, the device comprising:
at least one reactive material which undergoes a spectral change upon exposure to a pre-determined chemical compound; and
a support structure supporting the at least one reactive material, the support structure being adapted for positioning the at least one reactive material within expected diffusion range of the pre-determined chemical compound from the suspected diseased tissue.

2. A device as in claim 1, wherein the chemical compound is in a gaseous state at 37° C.

3. A device as in claim 1, wherein the chemical compound comprises nitric oxide and/or nitrogen dioxide.

4. A device as in claim 1, wherein the at least one reactive material comprises an NO-reactive material.

5. A device as in claim 1, wherein the at least one reactive material comprises a color-changing material.

6. A device as in claim 1, wherein the at least one reactive material comprises a luminescence-changing material.

7. A device as in claim 1, wherein the at least one reactive material is incorporated within the support structure.

8. A device as in claim 6, wherein the support structure comprises plastic having the at least one reactive material incorporated therein.

9. A device as in claim 1, wherein the at least one reactive material is attached to an external surface of the support structure.

10. A device as in claim 1, further comprising an external covering having the at least one reactive material, wherein the external covering covers at least a portion of the support structure.

11. A device as in claim 10, wherein the external covering comprises a coating, sheath or sleeve.

12. A device as in claim 1, wherein the support structure comprises a probe having an elongate tip adapted for insertion into an orifice of the patient leading to the suspected diseased tissue.

13. A device as in claim 12, wherein the tip is adapted for insertion into an anus and positioning of the at least one reactive material in a rectum, colon, small intestine or large intestine.

14. A device as in claim 12, wherein the tip is adapted for insertion into a mouth and positioning of the at least one reactive material in an upper respiratory tract, esophagus or stomach.

15. A device as in claim 12, wherein the tip is adapted for insertion into the mouth and positioning of the at least one reactive material near a tooth or between two teeth.

16. A device as in claim 1, wherein the support structure comprises a tethered body having a retrieving element, wherein the tethered body is adapted for positioning within the patient.

17. A device as in claim 16, wherein the tethered body comprises a plug configured for insertion within a rectum or vagina.

18. A device as in claim 16, wherein the tethered body comprises a gastric capsule configured for insertion within a stomach.

19. A device as in claim 1, wherein the support structure comprises at least one bead swallowable by the patient.

20. A device as in claim 19, wherein each bead has a magnetic core.

21. A device as in claim 1, wherein the support structure comprises a sheet adapted for positioning against a surface of the suspected diseased tissue.

22. A device as in claim 21, wherein the surface comprises skin of the patient.

23. A device as in claim 1, further comprising an external covering adapted for covering the at least one reactive material, wherein the external covering reduces leaching of the at least one reactive material.

24. A device as in claim 1, wherein the reactive material includes an energy transferring material.

25. A method of evaluating a suspected diseased tissue of a patient, the method comprising:
positioning a reactive material within an expected diffusion range of a pre-determined chemical compound from the suspected diseased tissue of the patient, wherein the reactive material is able to undergo a spectral change upon exposure to the pre-determined chemical compound;
observing the spectral change or an absence of the spectral change;
evaluating the suspected diseased tissue based on the observing step.

26. A method as in claim 25, wherein in response to observing the spectral change the method further comprises comparing the spectral change to a calibration to quantify the spectral change.

27. A method as in claim 25, wherein in response to observing the spectral change the method further comprises measuring the spectral change with an instrument.

28. A method as in claim 25, wherein the chemical compound comprises nitric oxide and/or nitrogen dioxide.

29. A method as in claim 25, wherein the reactive material comprises a color-changing material and wherein observing comprises determining a change in color or determining a lack of change in color.

30. A method as in claim 25, wherein the reactive material comprises a luminescence-changing material and wherein observing comprises determining a change in luminescence or a lack of change in luminescence.

31. A method as in claim 25, wherein positioning comprises inserting the reactive material into an orifice of the patient leading to the suspected diseased tissue.

32. A method as in claim 31, wherein inserting comprises inserting the reactive material into an anus.

33. A method as in claim 32, further comprising advancing the reactive material to a rectum, colon, small intestine or large intestine.

34. A method as in claim 31, wherein inserting comprises inserting the reactive material into a mouth.

35. A method as in claim 34, further comprising advancing the reactive material to an upper respiratory tract, esophagus or stomach.

36. A method as in claim 25, wherein the reactive material is supported by a support structure and wherein positioning comprises positioning the support structure near the suspected diseased tissue.

37. A method as in claim 36, wherein the support structure comprises a probe having an elongate tip carrying the reactive material and positioning comprises positioning the elongate tip near the suspected diseased tissue.

38. A method as in claim 36, wherein the support structure comprises a plug and positioning comprises positioning the plug within a rectum or vagina.

39. A method as in claim 36, wherein the support structure comprises a gastric capsule and positioning comprises positioning the capsule within a stomach.

40. A method as in claim 36, wherein the support structure comprises a bead and positioning comprises swallowing the bead.

41. A method as in claim 36, wherein the support structure comprises a sheet and positioning comprises positioning the sheet against a surface of the suspected diseased tissue.

42. A method as in claim 41, wherein the surface comprises skin of the patient.
